SEQUENCE LISTING (27 sequences)

SEQ ID NO: 1 - DNA, 1863 bp, Sorghum
  misc_feature: ACC Carboxyl Transferase domain sequence (Wild Type BTX430)
gcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccgattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtggttcccagattctgcaaccaagacagctcaggcattattagacttcaaccgtgaaggattgcctctgtttatcctggctaactggagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 2 - DNA, 1863 bp, Artificial Sequence
  Synthetic Polynucleotide
  misc_feature: ACC1 (TGG to TGC)
gcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccaattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtgcttcccagattctgcaaccaagacagctcaggcattattagacttcaaccgtgaaggattgcctctgtttatcctggctaactggagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 3 - DNA, 1863 bp, Artificial Sequence
  Synthetic Polynucleotide
  misc_feature: ACC2 (TGG to TCG)
gcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccaattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtcgttcccagattctgcaaccaagacagctcaggcattattagacttcaaccgtgaaggattgcctctgtttatcctggctaactggagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 4 - DNA, 1863 bp, Artificial Sequence
  Synthetic Polynucleotide
  misc_feature: ACC3 (GCA to GTA)
gcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccaattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtcgttcccagattctgtaaccaagacagctcaggcattattagacttcaaccgtgaaggattgcctctgtttatcctggctaactggagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 5 - DNA, 1863 bp, Artificial Sequence
  Synthetic Polynucleotide
  misc_feature: ACC4 (TGG to TCG)
gcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccaattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtcgttcccagattctgtaaccaagacagctcaggcattattagacttcaaccgtgaaggattgcctctgtttatcctggctaactcgagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 6 - protein, 620 aa, Sorghum
  MISC_FEATURE: Sorghum Wild type CT domain sequence
ANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVWFPDSATKTAQALLDFNREGLPLFILANWRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 7 - protein, 620 aa, Artificial Sequence
  Synthetic Polypeptide
  MISC_FEATURE: ACC1 (W1999C)
ANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVCFPDSATKTAQALLDFNREGLPLFILANWRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 8 - protein, 620 aa, Artificial Sequence
  Synthetic Polypeptide
  MISC_FEATURE: ACC2 (W1999S)
ANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVSFPDSATKTAQALLDFNREGLPLFILANWRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 9 - protein, 620 aa, Artificial Sequence
  Synthetic Polypeptide
  MISC_FEATURE: ACC3 (A2004V)
ANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVWFPDSVTKTAQALLDFNREGLPLFILANWRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 10 - protein, 620 aa, Artificial Sequence
  Synthetic Polypeptide
  MISC_FEATURE: ACC4 (W2027S)
ANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVWFPDSATKTAQALLDFNREGLPLFILANSRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 11 - DNA, 24 bp, Artificial Sequence
  Synthetic Primer
  misc_feature: PCR primer 1F
gcaactctggtgctaggattggca

SEQ ID NO: 12 - DNA, 28 bp, Artificial Sequence
  Synthetic Primer
  misc_feature: PCR Primer 1R
gaacatagctgagccacctcaatatatt

SEQ ID NO: 13 - DNA, 24 bp, Artificial Sequence
  Synthetic Primer
  misc_feature: PCR Primer 2F
ggtggtcctaagatcatggcgacc

SEQ ID NO: 14 - DNA, 26 bp, Artificial Sequence
  Synthetic Primer
  misc_feature: PCR Primer 2R
agtcttggagttcctctgacctgaac

SEQ ID NO: 15 - DNA, 23 bp, Artificial Sequence
  Synethic Primer
  misc_feature: PCR Primer 3F
cagcttgattcccatgagcgatc

SEQ ID NO: 16 - DNA, 25 bp, Artificial Sequence
  Synthetic Primer
  misc_feature: PCR Primer 3R
ccatacagtcttggagttcctctga

SEQ ID NO: 17 - DNA, 26 bp, Artificial Sequence
  Synthetic Polynucleotide
gagtgttatgctgagaggactgccaa

SEQ ID NO: 18 - DNA, 24 bp, Artificial Sequence
  Synthetic Primer
  misc_feature: PCR Primer 4R
accaaggaccttcttgacttcctg

SEQ ID NO: 19 - DNA, 17 bp, Artificial Sequence
  Synthetic Primer
  misc_feature: X (HAX dye)
gggctggacaagtgtgg

SEQ ID NO: 20 - DNA, 17 bp, Artificial Sequence
  Synthetic Primer
  misc_feature: Y (FAM dye)
gggctggacaagtgtgc

SEQ ID NO: 21 - DNA, 20 bp, Artificial Sequence
  Synthetic Primer
  misc_feature: C
ctgagctgtcttggttgcag

SEQ ID NO: 22 - DNA, 18 bp, Artificial Sequence
  Synthetic Primer
  misc_feature: X (HAX dye)
ttgcagaatctgggaacc

SEQ ID NO: 23 - DNA, 18 bp, Artificial Sequence
  Synthetic Primer
  misc_feature: Y (FAM dye)
ttgcagaatctgggaacg

SEQ ID NO: 24 - DNA, 20 bp, Artificial Sequence
  Synthetic Primer
  misc_feature: C
ggtcagcttgattcccatga

SEQ ID NO: 25 - DNA, 21 bp, Artificial Sequence
  Synthetic Primer
  misc_feature: X (HAX dye)
gtccaccagagaaacctctcc

SEQ ID NO: 26 - DNA, 21 bp, Artificial Sequence
  Synthetic Primer
  misc_feature: Y (FAM dye)
gtccaccagagaaacctctcg

SEQ ID NO: 27 - DNA, 20 bp, Artificial Sequence
  Synthetic Primer
  misc_feature: C
cgtgaaggattgcctctgtt

(19) United States
(12) Patent Application Publication
(10) Pub. No.: US 2022/0135993 A1
(43) Pub. Date: May 5, 2022

(54) MUTATIONS CONFERRING ACETYL-COA CARBOXYLASE (ACC) INHIBITING HERBICIDE TOLERANCE IN SORGHUM

(71) Applicant: S&W SEED COMPANY, Longmont, CO (US)

(72) Inventors: Rangaraj Nandakumar, Lubbock, TX (US); Song Luo, Chicago, IL (US); Scott A. Staggenborg, Lubbock, TX (US)

(21) Appl. No.: 17/648,697

(22) Filed: Jan. 24, 2022

Related U.S. Application Data
(63) Continuation of application No. 15/993,081, filed on May 30, 2018.
(60) Provisional application No. 62/513,074, filed on May 31, 2017.

(51) Int. Cl. C12N 15/82; C12N 9/00; A01H 6/46; A01H 1/04

(52) U.S. Cl. CPC C12N 15/8274; C12N 9/93; C12Y 604/01002; C12N 15/8206; A01H 1/04; A01H 6/4666

(57) ABSTRACT

The invention provides for Sorghum plants and plant parts developed through tissue culture, gene editing, or other methods of mutagenesis in which the plant or plant parts have increased tolerance to one or more acetyl-CoA carboxylase (ACC) herbicides at levels that would normally inhibit the growth of wild-type Sorghum plants. In this context, the Sorghum plant may be tolerant to any herbicide capable of inhibiting acetyl-CoA carboxylase enzyme activity. The present invention allows for the screening of ACC herbicide tolerant hybrids with markers or application of ACC inhibiting herbicides, and for the removal of unwanted vegetation with application of ACC inhibiting herbicides from seed and grain production fields.

This application is a Continuation Application of U.S. Ser. No. 15/993,081, filed May 30, 2018, which application claims priority to U.S. Provisional Patent Application No. 62/513,074, filed May 31, 2017, which is incorporated by reference herein in its entirety.

### INCORPORATION BY REFERENCE OF SEQUENCE LISTING

This application includes, as a separate part of the disclosure, a Sequence Listing in computer-readable form (filename: 52092 Seqlisting.txt; 44,910 bytes-ASCII text file; created May 24, 2018), which is incorporated by reference herein in its entirety.

### FIELD OF INVENTION

Sorghum plants and plant parts developed through tissue culture, gene editing or other methods of mutagenesis in which the plant or plant parts have increased resistance to one or more acetyl-CoA carboxylase (ACC) herbicides at levels that would normally inhibit the growth of wild-type Sorghum plants. In this context, the Sorghum plant may be tolerant to any herbicide capable of inhibiting acetyl-CoA carboxylase enzyme activity. For example, the Sorghum plant may be tolerant to herbicides of the aryloxyphenoxypropionate (FOP), cyclohexanedione (DIM) and phenylpyrazolin (DENs) herbicide family. This invention allows for creation of ACC herbicide resistant lines and hybrid seed through tissue culture or transgenic methods including gene editing methods, with up to 25 or 50% efficiencies of regenerating desired plants. The present invention allows for the screening of ACC herbicide resistant hybrids with markers or application of ACC inhibiting herbicides, and for the removal of unwanted vegetation with application of ACC inhibiting herbicides from seed and grain production fields.

### BACKGROUND

Sorghum is the second most important cereal-feed grain grown in the United States. Production is economically critical to farms operating in marginal rainfall areas because of Sorghum's ability to tolerate drought and heat. Both the livestock and bio-energy industries utilize Sorghum as an energy substrate thereby making it a versatile crop.

Sorghum is more tolerant to drought and excess soil moisture content than most cereals. It is capable of growing properly under varied soil and weather conditions. Likewise, it responds favorably to irrigation, requiring a minimum of 250 mm during its life cycle, with an optimum irrigation ranging from 400-550 mm.

Furthermore, Sorghum has the ability of remaining dormant during periods of drought and resumes growth under favorable periods, although these stress situations may affect performance.

Worldwide, Sorghum is the fifth leading cereal grain. As it is tolerant to both drought and heat, it is easily the most widely grown food grain in the semiarid regions of sub-Sahelian Africa and in the dry central peninsular region of India. As such, Sorghum is used in human consumption in most of the driest regions of the world thereby making it a critically important food crop in these locations.

The development of herbicide resistance in plants offers significant production and economic advantages; as such the use of herbicides for controlling weeds or plants in crops has become almost a universal practice. However, application of such herbicides can also result in death or reduced growth of the desired crop plant, making the time and method of herbicide application critical or in some cases unfeasible.

Of particular interest to farmers is the use of herbicides with greater potency, broad weed spectrum effectiveness and rapid soil degradation. Plants, plant tissues and seeds with resistance to these compounds would provide an attractive solution by allowing the herbicides to be used to control weed growth, without risk of damage to the crop. ACC herbicides are those with the mode of action that affect the acetyl-CoA carboxylase enzyme in the plant. This class of herbicide is only effective in controlling member of the Poaceae or Gramineae family of plants. Such herbicides are included in the aryloxyphenoxypropionate (FOP), cyclohexanedione (DIM) and phenylpyrazolin (DENs) chemical families. For example, Sorghum is susceptible to many ACC inhibiting herbicides that target monocot species, making the use of these herbicides to control grassy weeds almost impossible with conventional Sorghum hybrids and open pollinated varieties.

Certain weed grass species have been found that display altered sensitivity to FOP and DIM herbicides. One grass species, black grass (A. myosuroides [Huds.]), is a major grass weed in Europe. Several mutations have been found in the genome of some black grass plants that confer resistance to some, but not all, FOP and DIM herbicides (Délye, et al., 2005, Plant Phys. 137:794-806; Délye, et al., 2002, Theor. Appl. Genet. 104:1114-1120). Similar findings were found in mutant grass weeds such as annual ryegrass (L. rigidum [Gaud.]; Délye, et al., 2002, Pest Manag. Sci. 58:474-478), green foxtail (S. viridis [L. Beauv.]; Zhang and Devine, 2000, Weed Sci. Soc. Am. 40:33; Délye, et al., 2002, Planta 214:421-427) and wild oat (A. fatua [L.]; Christoffers et al., 2002, Genome 45:1049-1056). One herbicide resistant maize hybrid (DK592 from Dekalb) has a similar mutation in the ACC enzyme as that found in grass weeds (Zagnitko et al., 2001, Proc. Natl. Acad. Sci. 98:6617-22).

Creating mutations as means of altering a plants phenotype and composition is a common modern plant improvement practice. This can be accomplished through chemical or DNA damaging mutagenesis, gene editing, or through tissue culture selection. Chemical mutagenesis is the process where plant tissue, normally seeds, are exposed to a mutagen inducing compounds like ethyl methanesulfonate (EMS), sodium azide (AZ), or methyl nitrosoureas (MNU), or irradiated with X-rays, fast neutrons, or other types of DNA damaging particles. Mutagenized seed is then planted and the desired mutation is selected through a variety of methods, such as exposing plants from the mutagenized seed to an herbicide for which a resistant or tolerant plant is desired.

Creating mutations via tissue culture occurs by exposing undifferentiated cells, in callus, to the stress of choice in gradually increasing intensities to allow mutations to occur in the callus cells. The cells that mutate and survive the exposure and propagate and are exposed to gradually higher levels of intensity of the stress until a desired level is achieved. At this point, the callus must be transformed into a plant and propagated from seed, in the case of crop plants. Targeted genome editing is useful for creating plant traits and phenotypes, as well as for plant breeding. Multiple gene editing technologies were developed in the past years, including zinc finger nucleases (ZFNs), transcription activator-like effector nucleases (TALENs), and clustered regularly interspaced short palindromic repeat (CRISPR)/CRISPR-associated protein (Cas) systems, such as Cas9, Cas3, Cas8a, Cas5, Cas8b, Cas8c, Cas10d, Cse1, Cse2, Csy 1, Csy2, Csy3, GSU0054, Cas 10, Csm2, Cmr5, Cas 11, Csx10, Csf1, Csn2, Cas4, Cpf1, C2c1, C2c3, Cas13a, Cas13b, and Cas1. These editing platforms allow for reverse genetics, genome engineering and targeted transgene integration by inducing DNA double strands breaks in the specific genomic loci of a cell and then harnessing the cell's natural repair pathways.

One of the distinct advantages of creating mutations with either mutagenesis, gene editing, or editing, or tissue culture is that the trait of interest, herbicide tolerance in the case here, can be developed in elite breeding germplasm. This approach results in more stable breeding germplasm than selecting for similar mutations in wild relatives as was the case in Tuinstra and Al-Khatib (2017).

Three significant inventions exist in the area of Sorghum herbicide tolerance. Trucillo et al. (WO2013149674A1, WO2013149674A8) used chemical mutagenesis to create mutations to create Sorghum germplasm that inhibits AHAS enzyme activity, inferring tolerance to imidazolinone herbicides. Tuinstra and Al-Khatib (US9617530) created elite Sorghum germplasm that contained altered acetyl-CoA carboxylase (ACC) genes that inferred resistance to acetyl-CoA carboxylase herbicides which are herbicides from the aryloxyphenoxypropionate (FOP) and cyclohexanedione (DIM) chemical families. Tuinstra and Al-Khatib (WO2008073800AS) also created elite Sorghum germplasm that contained altered acetolactate synthase (ALS) genes and proteins that are resistant to inhibition by herbicides that normally inhibit the activity of the ALS protein. Both Tuinstra inventions were developed by screening wild Sorghum relatives that contained the altered genes, respectively.

Acetyl-CoA carboxylase (ACC) is a biotinylated enzyme that catalyzes the carboxylation of acetyl-CoA to produce malonyl-CoA. This carboxylation is a two-step, reversible reaction consisting of the ATP-dependent carboxylation of the biotin group on the carboxyl carrier domain by biotin-carboxylase activity followed by the transfer of the carboxyl group from biotin to acetyl-CoA by carboxyl-transferase activity (Nikolau et al., 2003, Arch. Biochem. Biophys. 414:211-22). Acetyl-CoA carboxylase is not only a key enzyme in plants for biosynthesis of fatty acids, a process that occurs in chloroplasts and mitochondria, but ACC also plays a role in the formation of long-chain fatty acids and flavonoids, and in malonylation that occurs in the cytoplasm. There are two isoforms of ACC with the chloroplastic ACC accounting for more than 80% of the total ACC activity (Herbert et al., 1996, Biochem. J. 318:997-1006). Aryloxyphenoxypropionate (FOP) and cyclohexanedione (DIM) are two classes of chemicals that are known to selectively inhibit chloroplastic ACC in grasses (Rendina et al., 1990, J. Agric. Food Chem. 38:1282-1287).

Due to the importance of Sorghum as a crop plant on the world stage, what are needed are Sorghum hybrids that are resistant to the inhibitory effects of ACC herbicides, thereby allowing for greater crop yield when these herbicides are used to control grassy weeds.

The present invention provides for compositions and methods for producing Sorghum plants, seeds, and plant parts that have modified acetyl-CoA carboxylase (ACC) genes and proteins that make these plants, seeds, and plant parts resistant to inhibition by herbicides that normally inhibit the activity of the ACC protein.

### SUMMARY OF INVENTION

The invention provides for ACC inhibitor herbicide tolerant Sorghum plants or plant parts thereof comprising one or more mutations of the Acetyl-CoA Carboxylase (ACC) gene, wherein the Sorghum plant or plant part has increased resistance to one or more ACC inhibiting herbicide as compared with a wild-type Sorghum cultivar or plant. The nucleotide sequence of the wild-type (un modified) ACC Sorghum gene is set out as SEQ ID NO: 1.

The ACC inhibitor herbicide tolerant Sorghum plants or plant parts of the invention include plant or plant parts corresponding to the deposit under ATCC Accession No. PTA-125106, PTA-125107 or PTA-125108, deposited on May 9, 2018 with the American Type Tissue Culture Collection (ATCC). The invention also provides for plant progeny of these ACC inhibitor herbicide tolerant Sorghum plants.

The invention also provides for seed corresponding to the deposit under ATCC Accession No. PTA-125106, PTA-125107 or PTA-125108, deposited on May 9, 2018 with the American Type Tissue Culture Collection (ATCC). The invention also provides for plant progeny of resulting from the seed.

In an embodiment of the invention, the ACC inhibitor herbicide tolerant Sorghum plants or plant parts thereof comprising one or more mutations of the Sorghum ACC gene, wherein the nucleotide sequence encoding the CT domain of the ACC protein comprises one of the following: the nucleotide sequence of SEQ ID NO: 2; the nucleotide sequence of SEQ ID NO: 3; the nucleotide sequence of SEQ ID NO: 4; the nucleotide sequence of SEQ ID NO: 5; the nucleotide sequence of SEQ ID NO: 2 and one of the following: the nucleotide sequence of SEQ ID NO: 3, SEQ ID NO: 4 or SEQ ID NO: 5; the nucleotide sequence of SEQ ID NO: 3 and one of the following: the nucleotide sequence of SEQ ID NO:4 or SEQ ID NO: 5; the nucleotide sequence of SEQ ID NO:4 and SEQ ID NO: 5; the nucleotide sequence of SEQ ID NO: 2, SEQ ID NO: 3 and SEQ ID NO:4; the nucleotide sequence of SEQ ID NO: 2, SEQ ID NO: 3 and SEQ ID NO: 5; the nucleotide sequence of SEQ ID NO: 2, SEQ ID NO: 4 and SEQ ID NO: 5; or The nucleotide sequence of SEQ ID NO: 3, SEQ ID NO: 4 and SEQ ID NO: 5 and wherein the Sorghum plant or plant part has increased resistance to one or more ACC inhibiting herbicide as compared with a wild-type Sorghum cultivar or plant. The ACC inhibitor herbicide tolerant Sorghum plant parts may be an organ, tissue, cell or seed.

In another embodiment of the invention, the ACC inhibitor herbicide tolerant Sorghum plants or plant parts thereof comprising one or more mutations of the Sorghum ACC gene, wherein ACC gene encodes a Sorghum acetyl-CoA protein having a CT domain comprising one or more of the following mutations: a Tryptophan to Cysteine amino acid substitution at an amino acid position 1999 (W1999C; SEQ ID NO: 7) aligning with the amino acid sequence of SEQ ID NO: 6, or a Tryptophan to Serine amino acid substitution at an amino acid position 1999 (W19995; SEQ ID NO: 8) aligning with the amino acid sequence of SEQ ID NO: 6, or an alanine to valine amino acid substitution at an amino acid position 2004 (A2004V; SEQ ID NO: 9) aligning with the amino acid sequence of SEQ ID NO: 6, or a Tryptophan to Serine amino acid substitution at an amino acid position 2027 (W2027S; SEQ ID NO: 10) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and a Tryptophan to Serine amino acid substitution at an amino acid position 1999 (W1999S; SEQ ID NO: 8) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and an alanine to valine amino acid substitution at an amino acid position 2004 (A2004V; SEQ ID NO: 9) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and a Tryptophan to Serine amino acid substitution at an amino acid position 2027 W2027S; SEQ ID NO: 10) aligning with the amino acid sequence of SEQ ID NO: 6, or a Tryptophan to Serine amino acid substitution at an amino acid position 1999 (W1999S; SEQ ID NO: 8) aligning with the amino acid sequence of SEQ ID NO: 6 and an alanine to valine amino acid substitution at an amino acid position 2004 (A2004V; SEQ ID NO: 9) aligning with the amino acid sequence of SEQ ID NO: 6, or a Tryptophan to Serine amino acid substitution at an amino acid position 1999 (W1999S; SEQ ID NO: 8) aligning with the amino acid sequence of SEQ ID NO: 6 and a Tryptophan to Serine amino acid substitution at an amino acid position 2027 (W2027S; SEQ ID NO: 10) aligning with the amino acid sequence of SEQ ID NO: 6, or an alanine to valine amino acid substitution at an amino acid position 2004 (A2004V; SEQ ID NO: 9) aligning with the amino acid sequence of SEQ ID NO: 6 and a Tryptophan to Serine amino acid substitution at an amino acid position 2027 (W2027S; SEQ ID NO: 10) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and a Tryptophan to Serine amino acid substitution at an amino acid position 1999 (W1999S) aligning with the amino acid sequence of SEQ ID NO: 6 and an alanine to valine amino acid substitution at an amino acid position 2004 (A2004V; SEQ ID NO: 9) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and a Tryptophan to Serine amino acid substitution at an amino acid position 1999 (W1999S; SEQ ID NO: 8) aligning with the amino acid sequence of SEQ ID NO: 6 and a Tryptophan to Serine amino acid substitution at an amino acid position 2027 (W20275; SEQ ID NO: 10) aligning with the amino acid sequence of SEQ ID NO: 6, or a Tryptophan to Cysteine amino acid substitution at an amino acid position 1999 (W1999C; SEQ ID NO: 7) aligning with the amino acid sequence of SEQ ID NO: 6 and an alanine to valine amino acid substitution at an amino acid position 2004 (A2004V; SEQ ID NO: 9) aligning with the amino acid sequence of SEQ ID NO: 6 and a Tryptophan to Serine amino acid substitution at an amino acid position 2027 (W20275; SEQ ID NO: 10) aligning with the amino acid sequence of SEQ ID NO: 6, or a Tryptophan to Serine amino acid substitution at an amino acid position 1999 (W19995; SEQ ID NO: 8) aligning with the amino acid sequence of SEQ ID NO: 6 and an alanine to valine amino acid substitution at an amino acid position 2004 (A2004V; SEQ ID NO: 9) aligning with the amino acid sequence of SEQ ID NO: 6 and a Tryptophan to Serine amino acid substitution at an amino acid position 2027 (W2027S; SEQ ID NO: 10) aligning with the amino acid sequence of SEQ ID NO: 6, wherein the Sorghum plant or plant part has increased resistance to one or more ACC inhibiting herbicide as compared with a wild-type Sorghum cultivar or plant. SEQ ID NO: 6 is the amino acid sequence of the wild-type ACC protein CT domain.

Any of the ACC inhibitor herbicide tolerant Sorghum plant or plant parts of the invention are tolerant or resistant to an aryloxyphenoxypropionate ACC inhibiting herbicide, a cyclohexanedione ACC inhibiting herbicide or a phenylpyrazolin ACC inhibiting herbicide. For example, the ACC inhibitor herbicide tolerant Sorghum plant or plant parts are tolerant or resistant when the ACC inhibiting herbicide is applied individually, or in an herbicide combination, at a level that inhibits growth of a wild type Sorghum plant. For example, the ACC inhibiting herbicide is clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop- ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8).

In some embodiments, the ACC inhibitor herbicide tolerant Sorghum plants or plant parts are homozygous or heterozygous for one or more mutations of the ACC gene provided in the present disclosure. Alternatively, the ACC inhibitor herbicide tolerant Sorghum plants or plant parts comprise one or more mutations of the ACC gene disclosed in the present disclosure in homozygous or heterozygous combinations.

In another embodiment, the invention provides for one or more Acetyl-CoA carboxylase (ACC) inhibiting herbicide capable of being used for controlling unwanted vegetation in one or more Sorghum growing areas, wherein the Sorghum plants in the growing area comprise one or more ACC inhibitor herbicide tolerant Sorghum plants provided in the present disclosure. For example, the Acetyl-CoA carboxylase (ACC) inhibiting herbicide include ACC inhibiting herbicide is clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12- 2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8).

In another embodiment, the invention provides for methods for creating an Acetyl-CoA carboxylase (ACC) inhibitor herbicide tolerant Sorghum plant or plant part having one or more mutations in the Acetyl-CoA Carboxylase (ACC) gene comprising the steps of: exposing a Sorghum plant or plant part to about 1 μM-200 μM of an ACC inhibitor herbicide, selecting a cell, plant or plant part which grows in the presence of up to 200 μM of an ACC inhibitor herbicide, and regenerating plant shoots from the selected cell, plant or plant part in the presence of an ACC inhibitor herbicide. In these methods, the Acetyl-CoA carboxylase (ACC) inhibiting herbicide is clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8).

The invention also provides for methods of creating an Acetyl-CoA carboxylase (ACC) herbicide tolerant Sorghum plant or plant part having one or more mutations in the Acetyl-CoA Carboxylase (ACC) gene, comprising the steps of a) mutating the endogenous nucleotide sequence encoding the ACC protein by inserting, deleting, modifying or replacing one or more nucleotides within the genome of living Sorghum tissue using an engineered nuclease that creates site-specific double-strand breaks (DSBs) at a desired location in the genome, b) selecting a cell, plant or plant part comprising the mutation and wherein the plant or plant part grows in the presence of up to 200 μM of an ACC inhibitor herbicide, and c) regenerating plant shoots from the selected cell, plant or plant part in the presence of an ACC inhibitor herbicide. For example, in any of these methods, the endogenous nucleotide sequence encoding the ACC protein is mutated using Meganuclease, Zinc-Finger Nuclease, TALEN, or CRISPR technologies.

In another embodiment, the invention provides for methods of creating an Acetyl- CoA carboxylase (ACC) herbicide tolerant Sorghum plant or plant part having one or more mutations in the Acetyl-CoA Carboxylase (ACC) gene, comprising the steps of a) transforming a plant cell with one or more expression vectors, wherein the expression vector comprises a transgene nucleotide sequence, wherein the transgene nucleotide sequence encodes a mutated ACC protein amino acid sequence, b) selecting a cell, plant or plant part that expresses the mutated ACC protein and grows in the presence of up to 200 μM of an ACC inhibitor herbicide, and c) regenerating plant shoots from the selected cell, plant or plant part in the presence of an ACC inhibitor herbicide. In any of the methods of the invention, the transgene nucleotide sequence is derived from any source.

In any of the method of the invention, the plant cells are transformed with any method known in the art. For example, the plant cell is transformed through PEG mediated protoplast transformation, protoplast electroporation, biolistics, or Agrobacterium mediated transformation. In addition, the biolistic transformation is biolistic using embryogenic callus.

In any of the method of the invention, which comprise a step of regenerating plant shoots from aselected cells, the plants shoots are regenerated at an efficiency of 25% or greater, 30% or greater, 35% or greater, 40% or greater, 45% or greater, 50% or greater or 60% greater. In addition, in any of the method of the invention which comprise a step of regenerating plant shoots from aselected cells, the efficiency of regenerating a Sorghum plant is 25% or greater, 30% or greater, 35% or greater, 40% or greater, 45% or greater, 50% or greater or 60% greater.

In any of the methods of the invention, the ACC inhibitor herbicide tolerant Sorghum plant or plant part comprises one or more of the mutations of the Acetyl-CoA carboxylase (ACC) gene disclosed herein.

In another embodiment, the invention provides for methods of producing ACC inhibitor herbicide tolerant Sorghum plant progeny comprising the steps of a) crossing a first ACC inhibitor herbicide tolerant Sorghum plant disclosed herein with a second Sorghum plant having a different genetic background, and b) selecting a progeny plant resulting from the crossing wherein the progeny comprises the mutation in the ACC gene of the first ACC inhibitor herbicide tolerant Sorghum plant. For example, the crossing step comprises transferring pollen from the first ACC inhibitor herbicide tolerant Sorghum plant to a wild-type Sorghum plant and said crossing results in a population of progeny plants comprising the mutation of the first ACC inhibitor herbicide tolerant Sorghum plant. Alternatively, the crossing step comprises planting sterile female Sorghum lines grown and pollen shedding Sorghum lines in isolated fields, wherein one or both of the Sorghum lines are ACC inhibitor herbicide tolerant Sorghum plants as disclosed herein, wherein the crossing results in hybrid seed comprising the mutation of the first ACC inhibitor herbicide tolerant Sorghum plant. In any of these methods, the progeny comprises one or more mutations of the Acetyl-CoA carboxylase (ACC) gene disclosed herein.

The invention also provides for methods of developing a population of Acetyl-CoA carboxylase (ACC) inhibitor herbicide tolerant Sorghum plants comprising the steps of a) screening a population of Sorghum plants to identify a plant comprising one or more of the mutations of the ACC gene disclosed herein , and b) propagating the identified Sorghum plants comprising a mutation in the ACC gene nucleotide sequence to develop a population of ACC inhibitor herbicide tolerant Sorghum plants. For example, the screening step comprises using DNA markers to identify the ACC inhibitor herbicide tolerant Sorghum plant or plant part. For example, the screening step comprises applying ACC inhibiting herbicides on the population of Sorghum plants. In any of these methods, the ACC inhibiting herbicides is applied using a spray carrier, wherein the herbicide is applied at two to four times the recommended rate of herbicide application per area of land or is applied at two to four times the herbicide concentration per volume of carrier. In addition, in any of these methods further comprise the step of selecting healthy plants 14 days after herbicide application to identify herbicide tolerant Sorghum plants.

In any of the methods of the invention which involve applying the ACC inhibiting herbicide, said herbicide is applied to one of the following: a segregating population of inbred lines in the field, greenhouse or growth chamber, wherein the resulting inbred lines are tolerant to ACC inhibiting herbicides and eliminate wild-type Sorghum plants; a field of sterile female A-lines, restorer male R-Lines, or both inbred lines in a breeding nursery where manual pollination or crossing is conducted for the production of hybrid seed, wherein the resulting hybrid seed is tolerant to ACC inhibiting herbicides and eliminate wild type Sorghum plants; a field or greenhouse containing both sterile female parent A-lines and restorer male parent R- Lines, or both parent inbred lines, wherein one or both parents are tolerant to ACC inhibiting herbicides, for production of hybrid seed that is tolerant to ACC inhibiting herbicides and to eliminate wild type Sorghum plants; or a grain production field in which hybrid (F1) seed was planted, wherein the seed is tolerant to ACC inhibiting herbicides to eliminate wild type Sorghum plants.

In any of these methods, the ACC inhibiting herbicides that is applied is clodinafop- propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7), haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2);, cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8). In particular, the ACC inhibiting herbicide is Quizalofop-p-ethyl or Clethodim or a mixture thereof, wherein the dose of Quizalofop-p-ethyl is equivalent to 6.3 g a.i/ha and the dose of Clethodim is equivalent to 12.5 g a.i/ha and the herbicide is applied to a segregating population of inbred lines in the field, greenhouse or growth chamber to create new ACC inhibiting herbicide tolerant inbred lines.

In another embodiment, the invention provides for a method of using an ACC inhibitor herbicide tolerant Sorghum plant or plant part of any one of claims 1-8, for the elimination of unwanted vegetation or for the production of seed or grain, comprising applying a mixture comprising one or more ACC inhibiting herbicides and a spray carrier, wherein the mixture is applied at a recommended rate of herbicide per area of land or concentration per volume of carrier for the control of weedy grasses in other tolerant crops. In any of these methods, the ACC inhibiting herbicide is clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8).

In particular, the ACC inhibiting herbicide is Quizalofop-p-ethyl, Clethodim or a mixture thereof, wherein the dose of Quizalofop-p-ethyl is equivalent to 6.3 g a.i/ha and the dose of Clethodim is equivalent to 12.5 g a.i/ha and the ACC inhibiting herbicide is applied to one of the following: a field or greenhouse containing sterile female parent A-lines, restorer male parent R-Lines, or both inbred parent lines, wherein one or both parents are tolerant to ACC inhibiting herbicides, for production of hybrid seed that is tolerant to ACC inhibiting herbicides; or a grain production field in which hybrid (F1) seed has been planted, wherein the hybrid seed is tolerant to ACC inhibiting herbicides for the purpose of controlling weeds and producing Sorghum grain or forage.

In another embodiment, the invention provides for methods for controlling unwanted vegetation in a Sorghum plant growing area comprising an ACC inhibitor herbicide tolerant Sorghum plant of any one of claims 1-8 with one or more ACC inhibitor herbicide(s), wherein the ACC inhibitor herbicide is applied alone or in combination with one or more non-ACC inhibitor herbicide. In these methods, the ACC inhibitor herbicide and the non-ACC inhibitor herbicide are applied jointly or simultaneously. Alternatively, the ACC inhibitor herbicide and the non-ACC inhibitor herbicide are applied at different times. In addition, in these methods the ACC inhibitor herbicide and the non-ACC inhibitor herbicide are applied sequentially, in pre- emergence applications followed by post-emergence applications, or in early post-emergence applications followed by medium or late post-emergence applications.

In any of these methods, the ACC herbicide(s) is clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); or pinoxaden (CAS RN 243973-20-8).

In any of the methods of the invention which comprise applying a non-ACC inhibiting herbicide, the non-ACC inhibiting herbicide is one of the following an inhibitor of lipid synthesis such as aryloxyphenoxypropionate, a cyclohexanedione, a benzofurane, a chloro-carbonic acid, a phosphorodithioate, a phenylpyrazolin or a thiocarbamate; an inhibitor of photosynthesis at photosystem II such as phenyl-carbamate, a pyridazinone, a triazine, a triazinone, a triazolinone, an uracil, an amide, an urea, a benzothiadiazinone, a nitrile or a phenyl-pyridine; an inhibitor of photosynthesis at photosystem I such as bipyridylium; an inhibitor of protoporphyrinogen oxidase such as diphenylether, a N-phenylphthalimide, an oxadiazole, an oxyzolidinedione, a phenylpyrazole, a pyrimidindione, or a thiadiazol; an inhibitor of carotenoid biosynthesis such as pyridazinone, a pyridinecarboxamide, an isoxazolidinone, or a triazole; an inhibitor of 4-hydroxyphenyl-pyruvate-callistemone such as isoxazole, a pyrazole, or a triketone; an inhibitor of EPSP synthase such as glycine; an inhibitor of glutamine synthesis such as phosphinic acid; an inhibitor of dihydropteroate synthase such as carbamate; an inhibitors of microtubule assembly such as benzamide, a benzoic acid, a dinitroaniline, a phosphoroamidate or a pyridine; an inhibitor of cell division such as acetamide, a chloroacetamide, or an oxyacetamide; an inhibitor of cell wall synthesis such as nitrile or a triazolocarboxamide; or an inhibitor of auxin transport such as a phthalamate or a semicarbazone.

### BRIEF DESCRIPTION OF DRAWING

FIG. 1 provides FOP herbicide resistant calli (FP-13) growing on tissue culture medium containing 1, 1.5, 2, 5, 10 μM of Quizalofop-p-ethyl and 5 μM of Assure II herbicide and

FIG. 2 provides FOP resistant calli (FP-7, FP-8, FP-9, FP-10, FP-13, FP-15) surviving on 100 μM of Quizalofop-p-ethyl (chemical AI.).

FIG. 3 demonstrates regeneration and rooting of FOP resistant (FP-13) and control BTX430 calli on media containing 0.0 and 1.0 μM Quizalofop-p-ethyl (chemical A.I.).

FIG. 4 provides DNA and protein sequences of the carboxyl transferase region of Sorghum ACC gene. SEQ ID NOS: 1 and 6 are DNA and protein sequences of wild type Sorghum (BTX430). SEQ ID NOS: 2 and 7, 3 and 8, 4 and 9, 5 and 10 represents the DNA and protein sequence of mutated ACC1, ACC2, ACC3 and ACC4, respectively with mutation at W1999C, W1999S, A2004V and W2027S codon position (W=Tryptophan, C=Cysteine, S=Serine, A=Alanine, V=Valine).

FIG. 5 provides a comparison of the four amino acid mutations at W1999C, W1999S, A2004V and W2027S in the CT domain of Sorghum ACC gene found to be associated with ACC herbicide resistance in Sorghum line BTX430-CHR-ACCs. (W=Tryptophan, C=Cysteine, S=Serine, A=Alanine, V=Valine).

FIG. 6 provides screening results of herbicide resistance in young (2 weeks old) tissue culture derived F₀ Sorghum plants (BTX430) containing W1999C mutation. Plants were sprayed with low rate of quizalofop herbicide Assure II at 2.5 oz/acre (0.5×), 5 oz/acre (1×) and 10 oz/acre (2×) and photographed 2 weeks after herbicide application. Arrow indicates tissue culture derived BTX430 control plants.

FIG. 7 provides screening results of herbicide resistance in young (2 weeks old) tissue culture derived F₀ Sorghum plants (FP11 and FP12) containing W1999S mutation. Plants were sprayed with low rate of quizalofop herbicide Assure II at 10 oz/acre (2×) and photographed 2 weeks after herbicide application. Arrow indicates tissue culture derived BTX430 control plants.

FIG. 8 provides screening results of herbicide resistance in young (2 weeks old) tissue culture derived F₀ Sorghum plants (FP5) containing W2027S mutation. Plants were sprayed with low rate of quizalofop herbicide Assure II at 10 oz/acre (2×) and photographed 2 weeks after herbicide application. Arrow indicates tissue culture derived BTX430 control plants.

FIG. 9 demonstrates herbicide resistance in matured (6-8 weeks old) tissue culture derived F₀ Sorghum plants (BTX430) containing W1999C mutation. Plants were sprayed with high field rate application of Assure II at 8 oz/acre (1×), 16 oz/acre (2×) and 32 oz/acre (4×) and photographed 3 weeks after herbicide application.

FIG. 10 demonstrates herbicide resistance in F₁ heterozygous Sorghum plants (BTX430) containing W1999C mutation. Plants were sprayed at 2× rate of Assure II (16 oz/acre) with spray volume of 15 gallons/acre. Plants were photographed 10 days after herbicide application.

FIG. 11 provides F₁ plant of BTX430 herbicide resistant plants (W1999C) with good seed set under greenhouse conditions.

FIG. 12 demonstrates KASP assay developed for detecting SNP at W1999C codon position of ACC gene. Three clusters separate homozygous mutants (TGC), heterozygous mutants (TG(G/C) and wild types allele (TGG).

FIG. 13A-FIG. 13B demonstrates complete herbicide resistance in homozygous F2 BTX430 plants with W1999C mutation. Picture shows before (A) and 14 days after (B) herbicide application at 2× rate (16 oz/acre).

FIG. 14A-FIG. 14B. Complete herbicide resistance in homozygous F₂ BTX430 plants with W1999C mutation. Pictures shows before (A) and 14 days after (B) herbicide application at 4× rate (32 oz/acre).

### DETAILED DESCRIPTION

As used herein, the term “variety” and “cultivar” refers to plants that are defined by the expression of the characteristics resulting from a given genotype or combination of genotypes, distinguished from any other plant grouping by the expression of at least one of the characteristics and considered as a unit with regard to its suitability for being propagation unchanged.

As used herein, the term “hybrid” refers to the offspring or progeny of genetically dissimilar plant parents or stock produced as the result of controlled cross-pollination, or by commercial hybrid seed production in which male and female lines are planted near to each other.

As used herein, the term “progeny” refers to generations or offspring of a plant.

As used herein, the term “derivative” of an herbicide resistant plant includes both the progeny of that herbicide resistant plant, as well as any mutant, recombinant, or genetically engineered derivative of that plant, whether of the same species or a different species, where the herbicide resistant characteristic(s) of the original herbicide resistant plant has been transferred to the derivative plant.

As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As used herein, the term “plant part” as used herein refers to a plant structure or a plant tissue, for example, pollen, an ovule, a tissue, a pod, a seed, a leaf, panicles, roots, caryopsis, stem and a cell. In some embodiments of the present invention transgenic plants are crop plants.

As used herein, the term “caryopsis” as used herein refers to a dry, single carpel and indehiscent fruit in which the ovary wall is united with the seed coat, typically of grass species. The caryopsis is commonly referred to as grain or seed, with the use often dependent upon the final use.

As used herein, the terms “crop” and “crop plant” are used in their broadest sense. The term includes, but is not limited to, any species of plant edible by humans or used as a feed for animal or fish or marine animal, or consumed by humans, or used by humans, or viewed by humans, or any plant used in industry, commerce, or education.

As used herein, the terms “F-generation” and “filial generation” refers to any of the consecutive generations of plants, cells, tissues or organisms after a biparental cross. The generation resulting from a mating of the a biparental cross (i.e. two parents) is the first filial generation (designated as “F₁” and “F₁”) in reference to a seed and it's plant, while that resulting from crossing of F₁ individual is the second filial generation (designated as “F₂” or “F₂”) in reference to a seed and it's plant. For example, an F₂ seed and a resulting plant are produced by self-pollination or cross-pollination of F₁, while later F generations are produced from self- pollination or cross-pollination of the immediate prior generation.

As used herein, the term “germplasm” refers to any genetic material of plants that contain functional units of heredity.

As used herein, the term “elite germplasm” in reference to a plant refers to hereditary material of proven genetic superiority.

As used herein, the term “elite plant,” refers to any plant that has resulted from breeding and selection for superior agronomic performance.

As used herein, the term “trait” refers to an observable and/measurable characteristic of an organism. For example, the present invention describes plants that are resistant to FOP and DIM herbicides, whereby that resistance is a trait.

As used herein, the terms “marker” and “DNA marker” and “molecular marker” in reference to a “selectable marker” refers to a physiological or morphological trait that may be determined as a marker for its own selection or for selection of other traits closely linked to that marker. For example, such a marker could be a gene or trait that associates with herbicide tolerance including, but not limited to, simple sequence repeat (SSR), single nucleotide polymorphism (SNP), genetic insertions and/or deletions and the like.

As used herein, the term “introgress” and “introgressing” and “introgression” refers to conventional (i.e. classic) pollination breeding techniques to incorporate foreign genetic material into a line of breeding stock. For example, the present invention provides for Sorghum crop plants introgressed with a mutant ACC gene for herbicide tolerance by crossing two plant generations.

As used herein, the term “herbicide tolerant” or “herbicide tolerance” refers to an improved capacity of a particular plant to withstand the various degrees of herbicidally induced injury that typically are lethal to wild-type plants of the same genotype at the same herbicidal dose. The term “herbicide resistant” or “herbicide resistance” refers to the inherited ability of a plant to survive and reproduce following exposure to a dose of herbicide normally lethal to the wild type. In a plant, resistance may be naturally occurring or induced by such techniques as genetic engineering or selection of variants produced by tissue culture or mutagenesis. As used herein, unless otherwise indicated, herbicide “resistance” is heritable and allows a plant to grow and reproduce in the presence of a typical herbicidally effective treatment by an herbicide for a given plant, as suggested by the current edition of The Herbicide Handbook as of the filing of the subject disclosure. As is recognized by those skilled in the art, a plant may still be considered “resistant” even though some degree of plant injury from herbicidal exposure is apparent. As used herein, the term “tolerance” or “tolerant” includes “resistance” or “resistant” plants as defined herein, as well an improved capacity of a particular plant to withstand the various degrees of herbicidally induced injury that typically are lethal in wild-type plants of the same genotype at the same herbicidal dose.

As used herein, the term “wild-type” when made in reference to a gene refers to a functional gene common throughout a plant population. A functional wild-type gene is that which is most frequently observed in a population and is thus arbitrarily designated the “normal” or “wild-type” form of the gene.

As used herein, the terms “modified” or “mutant” or “functional mutant” when made in reference to a gene or to a gene product refers, respectively, to a gene or to a gene product which displays modifications in sequence and/or functional properties (i.e., altered characteristics) when compared with the wild-type gene or gene product. Thus, the terms “modified” and “mutant” when used in reference to a nucleotide sequence refer to an nucleic acid sequence that differs by one or more nucleotides from another, usually related nucleotide acid sequence and the term “functional mutant” when used in reference to a polypeptide encodes by said “modified” or “mutant” nucleic acid refers to the protein or polypeptide that retains activity. In the present application, the ACC mutant protein, “or functional mutant” thereof is an ACC gene that retains its native activity to create essential amino acids. Additionally, a “modified” nucleotide sequence is interpreted as that found in the degenerate genetic code as known by those skilled in the art. For example, the genetic code is degenerate as there are instances in which different codons specify the same amino acid; a genetic code in which some amino acids may each be encoded by more than one codon. It is contemplated that the present invention may comprise such degeneracy (e.g., wherein a Sorghum hybrid comprises an ACC gene that is at least 70%identical or homologous, at least 80%identical or homologous, at least 85%identical or homologous, at least 90%identical or homologous, at least 95%identical or homologous, at least 97% identical or homologous, or at least 99% identical or homologous to the nucleotide sequence of SEQ ID NOS: 1-5 as found in, for example, the Sorghum germplasm. Despite the differences in the sequences the ACC genes of the invention, these ACC gene sequence retain the ability to confer resistance and/or tolerance to ACC inhibiting herbicides. The invention provides for these ACC nucleotide sequence that are homologous or exhibit sequence identity to SEQ ID NOS: 1-5, and comprise at least one of the mutations disclosed herein.

As used herein, the term “heterologous” when used in reference to a gene or nucleic acid refers to a gene that has been manipulated in some way.

As used herein, the term “portion” or “functional fragment” when used in reference to a protein (as in “a fragment of a given protein”, “a protein fragment”, a “portion of a protein”) refers to fragments of that protein. The fragments may range in size from four amino acid residues to the entire amino sequence minus one amino acid. In the present invention, the protein fragment is preferentially functional such that the protein fragment confers resistance to inhibition to ACC herbicides to a given plant.

When used herein, the term “transgenic”' means that a gene—which can be of the same or a different species—has been introduced via an appropriate biological carrier, like Agrobacterium tumefaciens or by any other physical means, like protoplast transformation or particle bombardment, into a plant and which gene is able to be expressed in the new host environment, namely the genetically modified organism (GMO).

As used herein, the term “gene editing” is a type of genetic engineering in which DNA is inserted, deleted, modified or replaced in the genome of a living organism. The common methods for such editing use engineered sequence-specific nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’).

In accordance to the before definition, the term “non-transgenic” means exactly the contrary, i.e. that no introduction of the respective gene has occurred via an appropriate biological carrier or by any other physical means. However, a mutated gene can be transferred through pollination, either naturally or via a breeding process to produce another non-transgenic plant concerning this specific gene.

An “endogenous gene” means a gene of a plant which has not been introduced into the plant by genetic engineering techniques.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used.

The Sorghum plants in the current invention show improved resistance to herbicides, for example herbicides targeting the ACC enzyme, such as aryloxyphenoxypropionates (FOP), cyclohexanediones (DIM) and phenylpyrazolins (DEN), as compared with wild-type Sorghum plants. In particular, the Sorghum plant (Sorghum bicolor) of the present invention comprises in its genome at least one polynucleotide encoding a polypeptide having a Tryptophan to Cysteine amino acid substitution at an amino acid position 1999 (SEQ ID NO: 7; W1999C) aligning with SEQ ID NO: 6; or a Tryptophan to Serine amino acid substitution at an amino acid position 1999 (SEQ ID NO: 8; W1999S) aligning with SEQ ID NO: 6; or an Alanine to Valine amino acid substitution at an amino acid position 2004 (SEQ ID NO: 9; A2004V) aligning with SEQ ID NO: 6; or a Tryptophan to Serine amino acid substitution at an amino acid position 2027 (SEQ ID NO: 10; W2027S) aligning with SEQ ID NO: 6 of Sorghum Acetyl-CoA Carboxylase large subunit.

The Sorghum plant may also comprise all possible combination of the mutations and subsequent amino acid substitutions shown in SEQ ID NOS: 2, 3, 4, and 5 and SEQ ID Nos. 7, 8, 9 and 10, respectively.

As such, the Sorghum plant may be tolerant to any herbicide or combination of herbicides capable of inhibiting ACC enzyme activity, i.e., the Sorghum plant may be tolerant to herbicides of the FOP family, such as, without limitation, Cyhalofop-butyl (CAS RN 122008-85-9); Diclofop-methyl (CAS RN 51338-27-3); Fenoxaprop-P-ethyl (CAS RN 71283-80-2); Fluazifop-P-butyl (CAS RN 79241-46-6); Quizalofop-P (CAS RN 76578-12-6); Haloxyfop (CAS RN 69806-34-4); Metamifop (CAS RN 256412-89-2); Propaquizafop (CAS RN 111479- 05-1), or herbicides from the DIM family, such as, without limitation, Clethodim (CAS RN 99129-21-2); Sethoxydim (CAS RN 74051-80-2); Tepraloxydim (CAS RN 149979-41-9); Tralkoxydim (CAS RN 87820-88-0), or herbicides from the DEN family, such as, without limitation, Pinoxaden (CAS RN 243973-20-8).

The “CAS RN” stated in parentheses behind the names corresponds to the “chemical abstract service registry number”, a customary reference number which allows the substances named to be classified unambiguously, since the “CAS RN” distinguishes, inter alia, between isomers including stereoisomers.

In a preferred embodiment, the Sorghum plant of the invention which is resistant to herbicides belonging to the groups FOP, DIM, or DEN herbicides is the Sorghum line designated BTX430-CHR-ACC1, BTX430-CHR-ACC2 and BTX430-CHR-ACC4 which seed were deposited with the ATCC, Manassas, Va., under Number PTA-125106, PTA-125108 and PTA-125107 respectively on May 9, 2018, under the terms of the Budapest Treaty. The mutated BTX430-CHR-ACC1 plant, parts thereof and it seeds, comprise in the genome mutated ACC gene comprising at least one polynucleotide encoding a polypeptide having a Tryptophan to Cysteine amino acid substitution at an amino acid position 1999 (SEQ ID NO: 7; W1999C) aligning with SEQ ID NO: 6 of Sorghum Acetyl-CoA Carboxylase large subunit. The mutated BTX430-CHR-ACC2 plant, parts thereof and it seeds, comprise in the genome mutated ACC gene comprising at least one polynucleotide encoding a polypeptide having a Tryptophan to Serine amino acid substitution at an amino acid position 1999 (SEQ ID NO: 8; W1999S) aligning with SEQ ID NO: 6 of Sorghum Acetyl-CoA Carboxylase large subunit. The mutated BTX430-CHR-ACC4 plant, parts thereof and it seeds, comprise in the genome mutated ACC gene comprising at least one polynucleotide encoding a polypeptide having a or a Tryptophan to Serine amino acid substitution at an amino acid position 2027 (SEQ ID NO: 10; W2027S) aligning with SEQ ID NO: 6 of Sorghum Acetyl-CoA Carboxylase large subunit.

In a preferred embodiment, the herbicide-resistant Sorghum plant comprises the resistance traits of BTX430-CHR-ACC1, BTX430-CHR-ACC2 and BTX430-CHR-ACC4 which seed were deposited with the ATCC, Manassas, VA, under Number PTA-125106, PTA-125108 and PTA-125107, and may be a plant as described in BTX430-CHR-ACC1, BTX430-CHR-ACC2 and BTX430-CHR-ACC4 which seed were deposited with the ATCC, a progeny of BTX430-CHR-ACC1, BTX430-CHR-ACC2 and BTX430-CHR-ACC4 which seed were deposited with the ATCC, Manassas, VA, under Number PTA-125106, PTA-125108 and PTA-125107, a mutant of BTX430-CHR-ACC1, BTX430-CHR-ACC2 and BTX430-CHR-ACC4 which seed were deposited with the ATCC, Manassas, VA, under Number PTA-125106, PTA-125108 and PTA-125107, and a progeny of BTX430-CHR-ACC1, BTX430-CHR-ACC2 and BTX430-CHR-ACC4 which seed were deposited with the ATCC, Manassas, Va., under Number PTA-125106, PTA-125108 and PTA-125107 mutant. Further provided is a Sorghum seed, comprising in its genome at least one polynucleotide encoding a polypeptide having a Tryptophan to Cysteine amino acid substitution at an amino acid position 1999 (SEQ ID NO: 7; W1999C) aligning with SEQ ID NO: 6; or a Tryptophan to Serine amino acid substitution at an amino acid position 1999 (SEQ ID NO: 8; W1999S) aligning with SEQ ID NO: 6; or an Alanine to Valine amino acid substitution at an amino acid position 2004 (SEQ ID NO: 9; A2004V) aligning with SEQ ID NO: 6; or a Tryptophan to Serine amino acid substitution at an amino acid position 2027 (SEQ ID NO: 10; W2027S) aligning with SEQ ID NO: 6 of Sorghum Acetyl-CoA Carboxylase large subunit. The seed germinates and produces a plant having increased resistance to one or more herbicides of the FOP, DIM, or DEN groups as compared with wild-type Sorghum plants. In a preferred embodiment said seed is the seed deposited as PTA-125106.

Sorghum plants are self-pollinating plants, but they can also be bred by cross-pollination. The development of Sorghum hybrids requires the development of pollinator parents (fertility restorers) and seed parent inbreds using the cytoplasmic male sterility-fertility restorer system, the crossing of seed parents and pollinator parents, and the evaluation of the crosses. Pedigree breeding programs combine desirable traits; in the present invention, the desirable trait being plant resistance to ACC herbicides. This trait is put into the breeding pool from one or more lines, such that new inbred lines can be created by crossing, followed by selection of plants with the desired trait, followed by more crossing, etc. New inbreds are crossed with other inbred lines (e.g., elite plant lines like those described herein).

Pedigree breeding starts with the crossing of two genotypes, in a preferred embodiment, the two genotypes might be BTX430-CHR-ACC1, and an elite Sorghum line (e.g., Chromatin Proprietary lines such as R.410, R.159, R.373) or BTX430-CHR-ACC2, and an elite Sorghum (e.g., Chromatin Proprietary lines such as R.410, R.159, R.373) or BTX430-CHR-ACC4, and an elite Sorghum line (e.g., Chromatin Proprietary lines such as R.410, R.159, R.373). BTX430-CHR-ACC1, BTX430-CHR-ACC2 and BTX430-CHR-ACC4 which seed were deposited with the ATCC, Manassas, Va., under Number PTA-125106, PTA-125108 and PTA-125107 on May 9, 2018. If the original two parents do not provide all of the desired characteristics, then other sources can be included in the breeding population. For example, if a hybrid is desired such that both ACC herbicide resistance and resistance to another herbicide group or insect resistance, such as resistance to white sugarcane aphid as described herein was desirous, then plants with both these attributes could be crossed using classical breeding techniques. In the pedigree method, superior plants are selfed and selected in successive generations. In the succeeding generations, the heterozygous condition gives way to homogeneous lines as a result of self-pollination and selection. Typically, in the pedigree method, five or more generations of selfing and selection are practiced (e.g., S₁, S₂, S₃, S₄, S₅, etc.).

Backcrossing is used to improve a plant line. Backcrossing transfers a specific desirable trait from one source to another that lacks the trait. This is accomplished by, for example, crossing a donor (e.g., BTX430-CHR-ACC1, BTX430-CHR-ACC2 or BTX430-CHR-ACC4) to an inbred line (e.g., an elite line as described herein). The progeny of this cross is then crossed back (i.e. backcrossing) to the elite inbred line, followed by selection in the resultant progeny for the desired trait (e.g., resistance to ACC herbicides). Following at least one, and as many as five or more, backcross generations with selection for the desired trait, the progeny are typically heterozygous for the locus (loci) controlling the desired phenotype, but will be like the elite parent for the other genetic traits. The last backcrossing then is typically selfed in order to give a pure breeding progeny for the gene being transferred.

In current hybrid Sorghum breeding programs, new parent lines are developed to be either seed-parent lines (e.g., Chromatin Proprietary B-lines B.791, B.1498, and B.230 or pollen-parent lines (e.g., Chromatin Proprietary R-lines R.410, R.M46, and R.373 depending on whether or not they contain fertility restoring genes; the seed-parent lines do not have fertility restoring genes and are male-sterile in certain cytoplasms (also known as “A” line plants) and male-fertile in other cytoplasms (also known as “B” line plants), whereas the pollen-parent lines are not male sterile and do contain fertility restoring genes (also known as “R” line plants). The seed-parent lines are typically created to be cytoplasmically male sterile such that the anthers are minimal to non-existent in these plants thereby requiring cross-pollination. The seed-parent lines will only produce seed, and the cytoplasm is transmitted only through the egg. The pollen for cross pollination is furnished through the pollen-parent lines that contain the genes necessary for complete fertility restoration in the F₁ hybrid, and the cross combines with the male sterile seed parent to produce a high-yielding single cross hybrid with good grain quality.

Typically, this cytoplasmic male sterility-fertility restorer system is performed for the production of hybrid seed by planting blocks of rows of male sterile (seed-parent) plants and blocks of rows of fertility restorer (pollen-parent) plants, such that the seed-parent plants are wind pollinated with pollen from the pollen-parent plant. This process produces a vigorous single-cross hybrid that is harvested and subsequently purchased or acquired and planted by the consumer. Male sterile, seed-parent plants can also be created by genetically breeding recessive male-sterile nuclear genes into a particular population; however, the cytoplasmic male sterility-fertility restorer system is typically the system used for breeding hybrid Sorghum. Sleper and Poehlman 2006, Breeding Field Crops, Fifth Ed., Blackwell Publishing, provides a good review of current Sorghum breeding procedures and is incorporated herein in its entirety.

In the present invention, hybrids resistant to herbicides such as, as but not limited to, ACC inhibiting herbicides, could be created by crossing parents in a production area where one or both parents contain the ACC herbicide resistance trait. These parents, or parent, thereby confer onto the resulting hybrid the herbicide resistance through genetic combination of the parent lines to the F₁ hybrid seed. The present invention is not limited to the elite parent Sorghum lines listed, and one skilled in the art will recognize that any elite Sorghum line would be equally amenable to the compositions and methods as described herein. The present invention is not limited to field production of hybrid seed, and one skilled in the art will recognize that hybrid seed production could be conducted in any environment where Sorghum plants can be grown and produce seed.

In the present invention, open pollinated Sorghum varieties (OPV) could be created that are resistant to, but not limited to, ACC inhibitor herbicides. Open pollinated varieties are different from inbred lines in that they are developed to be grown and the caryopses produced used as both grain and seed for future plant propagation. Grain uses may include, but are not limited to, animal feed, human food, ethanol production, and organic chemical production. Open pollinated variety breeding programs combine desirable traits; in the present invention, the desirable trait being plant resistance to ACC herbicides, by crossing a plant that contains the trait with an OPV, followed by selection of plants with the desired trait.

In one embodiment, the present invention provides a Sorghum germplasm that confers resistance to inhibition by ACC herbicides, singly or in conjunction with other pest resistance traits, for example insect tolerance to white sugarcane aphid (Melanaphis sacchari (Zehntner)) (J. S. Armstrong et al., J. of Econ Entomol. Vol 108, Pages 576-582, incorporated herein in its entirety). In some embodiments, for example, a Sorghum hybrid whose germplasm comprises a synthetic cryl Ac gene from Bacillus thuringiensis (Bt) is introgressed into a Sorghum line whose germplasm confers resistance to ACC herbicides. In some embodiments, for example, a Sorghum hybrid whose germplasm comprises a resistance to the fungal leaf disease Anthracnose (Colletotrichum graminicola) is introgressed into a Sorghum line whose germplasm confers resistance to ACC herbicides. As well, the incorporation of ACC herbicide resistance and insect resistance is accomplished via plant transgenesis into the same Sorghum hybrid. One skilled in the art will recognize the various techniques as described herein that are applicable to the incorporation of two or more resistance attributes into the same Sorghum plant.

In one embodiment, the present invention provides ACC herbicide resistance in Sorghum plants comprising a mutation in the ACC gene found in the germplasm from BTX430-CHR-ACC1 or BTX430-CHR-ACC2 or BTX430-CHR-ACC4, wherein the mutation is incorporated into elite Sorghum varieties through plant breeding and selection, thereby providing for the development of herbicide tolerant Sorghum crop hybrids that will tolerate the use of ACC inhibiting herbicides for weed control. Deployment of this herbicide tolerance trait in the aforementioned hybrids allows use of these herbicides to control monocot weeds that grow in the presence of these crops. In some embodiments, the incorporation of the ACC resistance germplasm into elite lines is via introgression, or classical breeding methods. In some embodiments, the incorporation of the ACC resistance gene into elite lines is via heterologous gene transgenesis. In some embodiments, the invention provides a Sorghum hybrid, wherein at least one ancestor of the Sorghum hybrid comprises an ACC resistant gene from germplasm designated BTX430-CHR-ACC1 or BTX430-CHR-ACC2 or BTX430-CHR-ACC4. In some embodiments, the ACC resistant herbicide gene comprises one or more of the mutations disclosed herein, for example the gene comprise at least one mutation described herein, or at least two mutations described herein or at least three mutations described herein. In addition, the ACC resistant herbicide gene is at least 70% homologous, at least 80% homologous, at least 85% homologous, at least 90% homologous, at least 95% homologous, at least 97% homologous, or at least 99% homologous to the ACC resistant herbicide gene as found in the germplasm BTX430-CHR-ACC1 or BTX430-CHR-ACC2 or BTX430-CHR-ACC4. In some embodiments, the ACC resistant herbicide gene is at least 70% homologous, at least 80% homologous, at least 85% homologous, at least 90% homologous, at least 95% homologous, at least 97% homologous, or at least 99% homologous to the ACC resistant herbicide gene as found in the germplasm BTX430-CHR-ACC1, BTX430-CHR-ACC2 or BTX430-CHR-ACC4, such as germplasm comprising a tryptophan to cysteine at amino acid position aligning with Trp₁₉₉₉ of the CT Domain of the ACC gene; or a tryptophan to serine amino acid substitution at an amino acid position aligning with Trp₁₉₉₉ of the CT domain of the ACC gene; or an alanine to valine at amino acid position aligning with Ala₂₀₀₄ of the CT Domain of the ACC gene; or a tryptophan to serine at amino acid position aligning with Trp₂₀₂₇ of the CT Domain of the ACC gene.

In another embodiment, the present invention provides ACC herbicide resistance in Sorghum plants comprising a mutation in the ACC gene found in the germplasm from BTX430-CHR-ACC1 or BTX430-CHR-ACC2 or BTX430-CHR-ACC4, wherein the mutation is incorporated into elite Sorghum varieties through gene editing technology, thereby providing for the development of herbicide tolerant Sorghum crop hybrids that will tolerate the use of ACC inhibiting herbicides for weed control. In some embodiments, the ACC resistant herbicide gene is at least 70% homologous, at least 80% homologous, at least 85% homologous, at least 90% homologous, at least 95% homologous, at least 97% homologous, or at least 99% homologous to the ACC resistant herbicide gene as found in the germplasm BTX430-CHR-ACC1 or BTX430- CHR-ACC2 or BTX430-CHR-ACC4. In some embodiments, the ACC resistant herbicide gene comprises one or more of the mutations disclosed herein, for example the gene comprise at least one mutation described herein, or at least two mutations described herein or at least three mutations described herein. In addition, the ACC resistant herbicide gene is at least 70% homologous, at least 80% homologous, at least 85% homologous, at least 90% homologous, at least 95% homologous, at least 97% homologous, or at least 99% homologous to the ACC resistant herbicide gene as found in the germplasm BTX430-CHR-ACC1 or BTX430-CHR-ACC2 or BTX430-CHR-ACC4, such as germplasm comprising a tryptophan to cysteine at amino acid position aligning with Trp₁₉₉₉ of the CT Domain of the ACC gene; or a tryptophan to serine amino acid substitution at an amino acid position aligning with Trp₁₉₉₉ of the CT domain of the ACC gene; or an alanine to valine at amino acid position aligning with Ala₂₀₀₄ of the CT Domain of the ACC gene; or a tryptophan to serine at amino acid position aligning with Trp₂₀₂₇ of the CT Domain of the ACC gene.

In some embodiments, ACC herbicide resistant germplasm is introgressed into an elite Sorghum line using classic breeding techniques. Examples of classical breeding methods for Sorghum can be found in, for example, Sleper and Poehlman, 2006, Breeding Field Crops, Fifth Edition, Blackwell Publishing, incorporated herein in its entirety.

In one embodiment, the ACC herbicide resistant germplasm is introgressed into a Sorghum plant that provides food for human consumption. In some embodiments, the ACC herbicide resistant germplasm is introgressed into Sorghum plants that provide food for livestock (e.g., poultry, cattle, swine, sheep, etc.). In some embodiments, the ACC herbicide resistant germplasm is introgressed into Sorghum plants that are used in industrial processes such as ethanol production, the production of organic chemicals or energy production from direct combustion of Sorghum plant materials. In one embodiment, the ACC herbicide resistant gene is introduced into the plant genome via transgenesis using vectors and technologies known in the art or by classical breeding.

In some embodiments, the present invention provides an ACC resistant germplasm of a Sorghum plant part of germplasm BTX430-CHR-ACC1 or BTX430-CHR-ACC2 or BTX430-CHR-ACC4, and said Sorghum plant part is one or more of a pollen, an ovule, a tissue, a pod, a seed, and a cell. In one embodiment, the present invention provides an F₁ hybrid whose germplasm comprises an ACC resistance gene as described herein. In some embodiments, the F₁ hybrid is a cross between two elite Sorghum lines, at least one of which contains a germplasm comprising an ACC resistance gene as described herein.

The present invention is not limited to Sorghum plants mutated with EMS. Within the scope of the present invention are Sorghum plants obtained by other mutation methods, for example methods such as radiation and chemical mutagens. Herbicide-resistant mutant plants can also be obtained by means of a process of selective pressure on cells cultured with an herbicide and selection of resistant cells to generate an herbicide-resistant plant. Details of mutation and breeding methods can be found in “Principles of Cultivar Development” Fehr, 1993, Macmillan Publishing Company, the disclosure of which is included herein by reference. Someone skilled in the art may also be able to create mutations by exposing seeds or callus to, but not limited to, the following: ion beams, cosmic radiation, x-ray radiation and gamma radiation.

Gene editing methods can also be used to develop mutations that creating traits with herbicide tolerant functions. Sequence-specific nuclease can be designed to target Sorghum ACCase carboxyltransferase domain (CT domain). Once the nuclease the double strand break on the target sequence in a living cell, the cell's DNA repair machinery will find the break and try to fix it. Mutations are created when the repair is not perfect. It could be a deletion or insertion by DNA repair through non-homology end joining (NHEJ) pathway. Alternatively, specific sequence modification (alteration) can be obtained when DNA is repaired through a homologous recombination (HR) pathway with a designed sequence (donor).

The approaches to modify the nucleotide sequence of the Sorghum ACC gene to confer ACC inhibiting herbicides tolerance disclosed herein are equally relevant and equally preferred. Any of the methods disclosed herein are contemplated to generate the ACC inhibitor herbicide tolerant Sorghum plant or plant part of the invention.

In one embodiment, the present invention provides use of a transgene comprising a heterologous gene such as a gene encoding a mutant ACC protein for providing the selected agronomic trait of ACC herbicide resistance. In one embodiment, the transgene comprises a mutant ACC gene as found in the germplasm designated BTX430-CHR-ACC1 or BTX430-CHR-ACC2 or BTX430-CHR-ACC4. In some embodiments, the transgene comprises one or more of the mutations disclosed herein. In addition, the transgene is at least 70% homologous, at least 80% homologous, at least 85% homologous, at least 90% homologous, at least 95% homologous, at least 97% homologous, or at least 99% homologous or is 100% identical to the ACC resistant herbicide gene as found in the germplasm BTX430-CHR-ACC1, or BTX430-CHR-ACC2 or BTX430-CHR-ACC4 (e.g., the nucleotide sequences set out as any one of SEQ ID NOS: 1-5). In some embodiments, the ACC resistant herbicide gene is at least 70% homologous, at least 80% homologous, at least 85% homologous, at least 90% homologous, at least 95% homologous, at least 97% homologous, or at least 99% homologous or is 100% identical to the ACC resistant herbicide gene as found in the germplasm BTX430-CHR-ACC1 or BTX430-CHR-ACC2 or BTX430-CHR-ACC4, such as germplasm comprising a tryptophan to cysteine at amino acid position aligning with Trp₁₉₉₉ of the CT Domain of the ACC gene; or a tryptophan to serine amino acid substitution at an amino acid position aligning with Trp₁₉₉₉ of the CT domain of the ACC gene(SEQ ID NO: 7 or 8; respectively), or an alanine to valine at amino acid position aligning with Alamo,' of the CT Domain of the ACC gene (SEQ ID NO: 9); or a tryptophan to serine at amino acid position aligning with Trp₂₀₂₇ of the CT Domain of the ACC gene (SEQ ID NO: 10).

Heterologous genes intended for expression in plants are first assembled in expression vectors containing a heterologous gene and appropriate transcriptional and translational control elements, methods of which are well known to those skilled in the art. Methods include in vitro recombinant DNA techniques, synthetic techniques, and in vivo genetic recombination. Exemplary techniques are widely described in the art (See e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y., herein incorporated by reference).

In general, these vectors comprise a nucleic acid sequence encoding a heterologous gene operably linked to a promoter and/or other regulatory sequences (e.g., enhancers, polyadenylation signals, etc.) required for expression in a plant.

Promoters include, but are not limited to, constitutive promoters, tissue-, organ-, and developmentally specific promoters, and inducible promoters. Examples of promoters include, but are not limited to; constitutive promoter 35S of cauliflower mosaic virus; a wound-inducible promoter from tomato, leucine amino peptidase (Chao et al., 1999, Plant Physiol 120:979-992, herein incorporated by reference); a chemically-inducible promoter from tobacco, Pathogenesis-Related 1 (induced by salicylic acid and benzothiadiazole-7-carbothioic acid S-methyl ester); a heat shock promoter (U.S. Pat. No. 5,187,267, herein incorporated by reference); a tetracycline-inducible promoter (U.S. Pat. No. 5,057,422, herein incorporated by reference); and seed- specific promoters.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters such as those disclosed herein. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (Odell et al., 1985, Nature 313:810; Rosenberg et al., 1987, Gene, 56:125; Guerineau et al., 1991, Mol. Gen. Genet. 262:141; Proudfoot, 1991, Cell, 64:671; Sanfacon et al., 1991, Genes Dev. 5:141 all of which are incorporated herein by reference).

In some embodiments, constructs for expression of the heterologous gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. Intron sequences have been routinely incorporated into plant transformation vectors, typically within the non-translated leader.

In some embodiments, a construct for expression of the heterologous nucleic acid sequence of interest also includes a regulator such as a nuclear localization signal (Kalderon et al., 1984, Cell 39:499; a plant translational consensus sequence (Joshi, 1987, Nucleic Acids Research 15:6643), an intron (Luehrsen and Walbot, 1991, Mol. Gen. Genet. 225:81), and the like, operably linked to the nucleic acid sequence encoding a heterologous gene.

In preparing the construct comprising the nucleic acid sequence encoding a heterologous gene, or encoding a sequence designed to decrease heterologous gene expression, various DNA fragments can be manipulated so as to provide for the DNA sequences in the desired orientation (e.g., sense or antisense) and, as appropriate, in the desired reading frame. For example, adapters or linkers can be employed to join the DNA fragments, or other manipulations can be used to provide for convenient restriction sites, removal of superfluous DNA, removal of restriction sites, and the like. For this purpose, in vitro mutagenesis, primer repair, restriction, annealing, resection, ligation, and the like is preferably employed, where insertions, deletions or substitutions (e.g., transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra, 1982, Gene 19: 259, the bar gene which confers resistance to the herbicide phosphinothricin (White et al., 1990. Nucl Acids Res. 18:1062), the hph gene which confers resistance to the antibiotic hygromycin (Blochlinger and Diggelmann, 1984, Mol. Cell. Biol. 4:2929, incorporated herein by reference), and the dhfr gene that confers resistance to methotrexate (Bourouis et al., 1983, EMBO J., 2:1099, incorporated herein by reference).

In some embodiments, the Ti (T-DNA) plasmid vector is adapted for use in an Agrobacterium mediated transfection process such as in U.S. Pat. No. 6,369,298 (Sorghum), and U.S. Pat. Nos. 5,981,839; 6,051,757; 5,981,840; 5,824,877; and 4,940,838, all of which are incorporated by reference herein in their entireties. Construction of recombinant Ti and Ri plasmids in general follows methods typically used with more common vectors, such as pBR322. Additional use can be made of accessory genetic elements sometimes found with the native plasmids and sometimes constructed from foreign sequences. These may include, but are not limited to, structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The use of T-DNA as a flanking region in a construct for integration into a Ti- or Ri-plasmid has been described in EPO No. 116,718 and PCT Application Nos. WO 84/02913, 02919 and 02920; Herrera-Estrella, 1983, Nature 303:209-213; Fraley et al., 1983, Proc. Natl. Acad. Sci, USA 80:4803-4807; all of which are herein incorporated by reference.

The second system is called the “binary” system in which two plasmids are used and the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

In some embodiments, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967 herein incorporated by reference). One of skill in the art knows that homologous recombination may be achieved using targeting vectors that contain sequences that are homologous to any part of the targeted plant gene, whether belonging to the regulatory elements of the gene or the coding regions of the gene. Homologous recombination may be achieved at any region of a plant gene so long as the nucleic acid sequence of regions flanking the site to be targeted is known. Agrobacterium tumefaciens is a common soil bacterium that causes crown gall disease by transferring some of its DNA to the plant host. The transferred DNA (T-DNA) is stably integrated into the plant genome, where its expression leads to the synthesis of plant hormones and thus to the tumorous growth of the cells. A putative macromolecular complex forms in the process of T-DNA transfer out of the bacterial cell into the plant cell.

In some embodiments, the nucleic acids as disclosed herein are utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted heterologous polynucleotide can be expressed from these vectors as a fusion protein (e.g., coat protein fusion protein) or from its own subgenomic promoter or another promoter. Methods for the construction and use of such viruses are described in U.S. Pat. Nos. 5,846,795; 5,500,360; 5,173,410; and 5,965,794, all of which are incorporated herein by reference.

In some embodiments, a heterologous nucleic acid sequence of interest comprising a mutant ACC transgene, for example, as found in the germplasm designated BTX430-CHR-ACC1 or BTX430-CHR-ACC2 or BTX430-CHR-ACC4, is introduced directly into a plant. In some embodiments, the transgene is at least 70% homologous, at least 80% homologous, at least 85% homologous, at least 90% homologous, at least 95% homologous, at least 97% homologous, or at least 99% homologous or 100% identical to the ACC resistant herbicide gene as found in the germplasm BTX430-CHR-ACC1 or BTX430-CHR-ACC2 or BTX430-CHR- ACC4 (e.g., any one of SEQ ID NOS: 2-5). In some embodiments, the transgene is at least 70% homologous, at least 80% homologous, at least 85% homologous, at least 90% homologous, at least 95% homologous, at least 97% homologous, or at least 99% homologous or 100% identical to the ACC resistant herbicide gene as found in the germplasm BTX430-CHR-ACC1 or BTX430-CHR-ACC2 or BTX430-CHR-ACC4, such as germplasm comprising a tryptophan to cysteine at amino acid position aligning with Trp₁₉₉₉ of the CT Domain of the ACC gene; or a tryptophan to serine amino acid substitution at an amino acid position aligning with Trp 1999 of the CT domain of the ACC gene(SEQ ID NO: 7 or 8; respectively), or an alanine to valine at amino acid position aligning with Alamo,' of the CT Domain of the ACC gene (SEQ ID NO: 9); or a tryptophan to serine at amino acid position aligning with Trp₂₀₂₇ of the CT Domain of the ACC gene (SEQ ID NO:10).

One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278, herein incorporated by reference).

Once a nucleic acid sequence encoding the heterologous gene is operatively linked to an appropriate promoter and inserted into a suitable vector for the particular transformation technique utilized (e.g., one of the vectors described above), the recombinant DNA described above can be introduced into the plant cell in a number of art-recognized ways. Those skilled in the art will appreciate that the choice of method depends on the type of plant targeted for transformation. In some embodiments, the vector is maintained episomally. In some embodiments, the vector is integrated into the genome. In some embodiments, direct transformation in the plastid genome is used to introduce the vector into the plant cell (for example, see U.S. Pat. Nos. 5,451,513 and 5,545,817, all of which are incorporated herein by reference in their entireties).

The basic technique for chloroplast transformation involves introducing regions of cloned plastid DNA flanking a selectable marker together with the nucleic acid encoding the sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., 1990, Proc. Natl. Acad. Sci. 87:8526); Staub and Maliga, 1992, Plant Cell, 4:39, all of which are incorporated herein by reference). The presence of cloning sites between these markers allows creation of a plastid targeting vector introduction of foreign DNA molecules (Staub and Maliga, 1993, EMBO J., 12:601). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga, 1993, Proc. Natl. Acad. Sci. 90:913). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of RNAs encoded by the DNA molecule.

In one embodiment, vectors useful in the practice of the present invention are microinjected directly into plant cells (Crossway, 1985, Mol. Gen. Genet, 202:179). In some embodiments, the vector is transferred into the plant cell by using polyethylene glycol (Krens et al., 1982, Nature, 296:72) fusion of protoplasts with other entities such as minicells, cells, lysosomes or other fusible lipid-surfaced bodies (Fraley et al., 1982, Proc. Natl. Acad. Sci. USA 79:1859); and protoplast transformation (EP 0 292 435); direct gene transfer (Paszkowski et al., 1984, EMBO J., 3:2717. In some embodiments, the vector may also be introduced into the plant cells by electroporation. (Fromm, et al., 1985, Proc. Natl. Acad. Sci. USA 82:5824; Riggs and Bates., 1986, Proc. Natl. Acad. Sci. USA 83:5602). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In addition to direct transformation, in some embodiments, the vectors comprising a nucleic acid sequence encoding a heterologous gene are transferred using Agrobacterium-mediated transformation (Hinchee et al., 1988, Nature Biotechnology, 6:915; Ishida et al., 1996, Nature Biotechnology 14:745, all of which are herein incorporated by reference). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Heterologous genetic sequences (e.g., nucleic acid sequences operatively linked to a promoter of the present invention) can be introduced into appropriate plant cells, by means of the Ti plasmid of Agrobacterium tumefaciens (previously described). The Ti plasmid is transmitted to plant cells on infection by Agrobacterium tumefaciens, and is stably integrated into the plant genome (Schell, 1987, Science, 237:1176). Species that are susceptible to infection by Agrobacterium may be transformed in vitro. Transformation methods for producing transgenic Sorghum plants using Agrobacterium-mediated transformation are provided in U.S. Pat. No. 6,369,298.

In some embodiments, the vector is introduced through ballistic particle acceleration (U.S. Pat. No. 4,945,050; Casas et al., 1993, Proc. Natl. Acad. Sci. USA 90:11212, all references are incorporated herein in their entireties).

In some embodiments, after selecting for transformed plant material that can express a heterologous gene encoding a heterologous protein or variant thereof, whole plants are regenerated. Plant regeneration from cultured protoplasts is described in Evans et al., Handbook of Plant Cell Cultures, Vol. 1: (MacMillan Publishing Co. New York, (1983); Vasil I. R. (ed.), Cell Culture and Somatic Cell Genetics of Plants, Acad. Press, Orlando, Vol. I, (1984) and Vol. III, (1986), incorporated herein by reference in their entireties. It is known that many plants can be regenerated from cultured cells or tissues including, but not limited to, all major species of sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins, and shoots and roots will normally develop simultaneously. Efficient regeneration will depend on the medium, on the genotype, and on the history of the culture. The reproducibility of regeneration depends on the control of these variables.

In some embodiments, after selecting for transformed plant material that can express a heterologous gene encoding a heterologous protein or variant thereof, whole plants are regenerated. Transformation efficiency is measured as the percentage of regenerated resistant events out of the total number of plant material used for transformation.

In some embodiments, transgenic plants were regenerated from green callus after ballistic particle acceleration (U.S. Pat. No. 6,486,384). High frequency Sorghum transformation procedure is developed. In some embodiments, the transformation efficiency is at least 25%, at least 30%, at least 35%, at least 40%, at least 45%, at least 50%, or greater than 50%. In some embodiments, the regenerated plants are resistant to ACCase inhibitor herbicide.

Herbicide tolerant mutations can be developed through gene editing technology. In some embodiments, sequence-specific nucleases used for gene editing can be ZFN, TALEN, CRISPR/Cas9. In some embodiments, ACCase mutations developed through gene editing can be but not limited to W1999C, W1999S, A2004V, W2027S.

Tissue culture, transformation and regeneration of Sorghum have been reported by several groups since it was first transformed in 1991. Although Sorghum has been successfully transformed by both Agrobacterium and biolistic methods, the efficiency of transformation is very low (<10%) causing Sorghum to be classified as a recalcitrant crop for both tissue culture and genetic transformation. Increasing the transformation efficiency to higher levels (as observed in other monocot crop plants such as corn, rice and sugarcane) would be valuable to more rapidly genetically improve the crop. An improved transformation system would reduce time, cost, and resources necessary to evaluate genetic elements in Sorghum and to generate transgenic plants. Many variables can influence Sorghum transformation efficiency. Use of an efficient selection scheme to identify events is a critical variable for success. Several selectable maker genes, including NptII, hptII, bar and pmi have previously been tested in Sorghum using a range of promoters (i.e. CaMV35S, riceActin1, maizeUbi1, maize ADH, etc.)

In some embodiments, Sorghum transformation efficiency could be improved by replacing the yeast-based promoter with a stronger plant promoter. In general, For for transforming Sorghum, callus materials were produced from immature Sorghum as described by Gurel et al., 2009. Calli are transferred to fresh media every three weeks until the entire callus has turned into compact and friable callus. Three-week-old calli are subcultured into 3-4 mm diameter pieces and placed on medium that contains compounds to maintain a prescribed osmotic level for 4 h prior bombardment. Approximately 30 calli were placed at the center of a Petri dish (15×90 mm) containing osmotic medium and stored for 4-5 hours under a prescribed light treatment prior to bombardment. Plasmid DNA, can be coated onto 0.6 μm diameter gold particles as described by Carlson et al., 2007 and delivered into calli using a gas-powered biolistic delivery system. Bombardment may be carried out with a system configuration as described by Carlson, et al, 2007. Following bombardment, the cultures are transferred onto medium and incubated under 28° C. for 4-6 days and then were transferred to medium containing a selective agent and cultured under long-days with fluorescent lighting for 14 days at 28° C. The surviving calli can selected with two higher concentration of a selective agent at 14 days interval. All the transgenic calli events can regenerated and developed into a whole plant.

In some embodiments, a method that employs a strong plant promoter in combination with other promoters, in a preferred embodiment, this other promoter would be NptII, could significantly outperform a yeast promoter and NptII combination. In some embodiments, the strong plant promoter transformation efficiency (TE) is expected to exceed 25% with TE levels potentially as high as 55%. This frequency puts Sorghum transformation on par with other important monocot crops. In addition to being more efficient in producing numbers of events, those events produced with strong plant promoter and NptII combination were also able to be identified approximately 2 weeks earlier than events produced with yeast promoter and NptII. The size (biomass) of the selected callus averaged 5-fold larger with stronger promoter and NptII than with yeast promoter and NptII, allowing the earlier identification and for scientists to regenerate more plants per event.

Segregating plant populations must be screened to identify progeny that contain desirable traits, resistance to ACC inhibiting herbicides in the present invention. Plants with desired traits can be screened via phenotypic and genotypic methods. In the present invention, phenotypic screening can be accomplished through the application of ACC inhibiting herbicides whereas genotypic screening can be accomplished via genetic markers.

In the present invention, marker identification of plants containing the desired trait could be accomplished through the use of marker which include, but not limited to, simple sequence repeat (SSR), single nucleotide polymorphism (SNP), genetic insertions and/or deletions. One embodiment of the present invention might employ Kompetitive Allele Specific PCR (KASP) DNA markers developed for all SNPs associated with the desired mutation.

One skilled in the art could employ KASP by a real time PCR allelic discrimination assay using a thermocycler and amplified PCR gels. The result of the allelic discrimination could be determined by endpoint detection using methods such as, but not limited to, fluorescence detection. Comparison of KASP assay results between DNA extracted from plants that might contain the mutation and a wild type allow for the confirmation of presence or absence of the desired mutation. This approach can be used on calli and plants.

Phenotypic screening could be accomplished through the application of herbicides from the family of the desired resistance trait; in the present invention resistance to ACC inhibiting herbicides is the desired trait. In the present invention, if plants from a segregating population created by one of the methods described above are exposed to herbicides from the FOP, DIM, or DEN herbicide groups, plants containing the desired mutation are expected to survive.

One skilled in the art will recognize that the appropriate application dose is important for developing plants with the appropriate level of resistance. In the present invention, plants that contain the mutations that result in plants that are resistant to ACC inhibiting herbicides could be detected by applying the recommend rate of an herbicide from the FOP, DIM, or DEN family based on the herbicide manufacturer's recommendation for controlling shattercane (Sorghum bicolor). In the present invention, the herbicide application rate might also be increased to twice the rate or might also be increased to four times the rate as recommended by the herbicide manufacturer for control of shattercane (Sorghum bicolor).

One skilled in the art will recognize that the herbicide application may occur at various stage of plant or cell development. In the present invention, the desired herbicide is from the FOP, DIM or DEN family, and could be applied to the media that is used to produce callus or other artificial plant propagation media.

Alternatively, the desired herbicide could be applied to irrigation water or hydroponic solutions used to propagate plants, or could be applied directly to the foliage of plants being grown in soil or other media in a field, greenhouse, or plant growth chamber. These plants may range in age from the presence of a single leaf collar to physiological maturity, which is identified by the presence of a black layer at the base of the mature caryopsis.

In one embodiment, the present invention provides methods for controlling weeds in a field of any ACC herbicide resistant Sorghum plants include hybrid Sorghum crop plants. In some embodiments, controlling the weeds comprises applying an ACC herbicide to said field of Sorghum plants, such that weed growth is inhibited but Sorghum growth is not adversely affected. In some embodiments, the ACC herbicide being applied is from the aryloxyphenoxypropionate (FOP), cyclohexanedione (DIM) and phenylpyrazolin (DENs) herbicide families including, but not limited to, clodinafop-propargyl (CAS RN 105512-06-9): cyhalofop-butyl (CAS RN 122008-85-9); diclofop-methyl (CAS RN 51338-27-3); fenoxaprop-p-ethyl (CAS RN 71283-80-2); fluazifop-P-butyl (CAS RN 79241-46-6); quizalofop-p-ethyl (CAS RN 100646-51-3); quizalofop-p (CAS RN 94051-08-8); haloxyfop (CAS RN 69806-34-4); haloxyfop-ethoxyethyl (CAS RN 87237-48-7); haloxyfop-etotyl (CAS RN 87237-48-7); haloxyfop-R-methyl (CAS RN 72619-32-0); metamifop (CAS RN 256412-89-2); propaquizafop (CAS RN 111479-05-1); alloxydim (CAS RN 55634-91-8); butroxydim (CAS RN 138164-12-2); cycloxydim (CAS RN 101205-02-1); clethodim (CAS RN 99129-21-2); profoxydim (CAS RN 139001-49-3); sethoxydim (CAS RN 74051-80-2); tepraloxydim (CAS RN 149979-41-9); tralkoxydim (CAS RN 87820-88-0); pinoxaden (CAS RN 243973-20-8). In some embodiments, the ACC herbicide being applied comprises a combination of compounds from both FOP and DIM ACC herbicide families as disclosed herein. However, the present application is not limited to the ACC herbicide used, and a skilled artisan will appreciate that new ACC herbicides are being discovered at any given time that inhibit the ACC enzyme.

As such, one embodiment of the present invention provides a Sorghum germplasm that contains altered ACC genes and proteins. In some embodiments, the present invention provides for the use of ACC herbicides in fields of hybrid Sorghum crop plants to reduce the amount of unwanted vegetation present in said crop field, wherein said hybrid Sorghum germplasm comprises an altered ACC enzyme that confers resistance to ACC herbicides and said weed plants are ACC herbicide susceptible.

As such, one embodiment of the present invention provides a Sorghum germplasm that contains altered ACC genes and proteins. In some embodiments, the present invention provides for the use of ACC herbicides in fields of hybrid Sorghum crop plants to reduce the amount of monocot weed plants present in said crop field, wherein said hybrid Sorghum germplasm comprises an altered ACC enzyme that confers resistance to ACC herbicides and said weed plants are ACC herbicide susceptible.

In one embodiment, the removal of unwanted vegetation from a Sorghum growing area that comprises the presence of one or more Sorghum plants that are resistant to ACC inhibiting herbicides and applying one or more ACC inhibitor herbicide(s) alone or in combination with one or more herbicide(s) that do(es) not belong to the class of ACC inhibitor herbicides (non-ACC inhibitor herbicides), and wherein the application of the herbicides as defined under takes place jointly or simultaneously, or takes place at different times and/or in a plurality of portions (sequential application). These applications may include pre-emergence applications followed by post-emergence applications or early post-emergence applications followed by medium or late post-emergence applications. However, the present application is not limited to the ACC herbicide used or the application timing or methods and a skilled artisan will appreciate that new ACC herbicides are being discovered at any given time that inhibit the ACC enzyme.

In one embodiment, the removal of unwanted vegetation, or weeds, could from be from areas where hybrid Sorghum seed is being produced from one or more parent lines (A-line or R-line) that is resistant to ACC herbicides. These areas could include greenhouse growing areas and vessels or production fields. However, the present application is not limited to the ACC herbicide used, and a skilled artisan will appreciate that new ACC herbicides are being discovered at any given time that inhibit the ACC enzyme.

In one embodiment, the removal of unwanted vegetation, or weeds, could from be from areas where Sorghum grain is being produced from one or more hybrids that are resistant to ACC herbicides. These areas could include greenhouse growing areas and vessels or end user grain production fields. However, the present application is not limited to the ACC herbicide used, and a skilled artisan will appreciate that new ACC herbicides are being discovered at any given time that inhibit the ACC enzyme.

In one embodiment, the present invention provides for an ACC herbicide resistant Sorghum plants or a Sorghum hybrid (e.g., F₁, F₂, F₃, F₄, etc.) whose germplasm confers resistance to ACC herbicides and resistance to one or more additional herbicides from one or more different herbicide groups. For example, additional herbicide groups used to inhibit weed growth, include, but are not limited to, inhibitors of lipid synthesis (e.g., aryloxyphenoxypropionates, cyclohexanediones, benzofuranes, chloro-carbonic acids, phosphorodithioates, thiocarbamates), inhibitors of photosynthesis at photosystem II (e.g., phenyl-carbamates, pyridazinones, triazines, triazinones, triazolinones, uracils, amides, ureas, benzothiadiazinones, nitriles, phenyl-pyridines), inhibitors of photosynthesis at photosystem I (e.g., bipyridyliums), inhibitors of protoporphyrinogen oxidase (e.g., diphenylethers, N-phenylphthalimides, oxadiazoles, oxyzolidinediones, phenylpyrazoles, pyrimidindiones, thiadiazoles), inhibitors of carotenoid biosynthesis (e.g., pyridazinones, pyridinecarboxamides, isoxazolidinones, triazoles), inhibitors of 4-hydroxyphenyl-pyruvate-dioxygenase (e.g., callistemones, isoxazoles, pyrazoles, triketones), inhibitors of EPSP synthase (e.g., glycines), inhibitors of glutamine synthetase (e.g., phosphinic acids), inhibitors of dihydropteroate synthase (e.g., carbamates), inhibitors of microtubule assembly (e.g., benzamides, benzoic acids, dinitroanilines, phosphoroamidates, pyridines), inhibitors of cell division (e.g., acetamides, chloroacetamides, oxyacetamides), inhibitors of cell wall synthesis (e.g., nitriles, triazolocarboxamides) and inhibitors of auxin transport (e.g., phthalamates, semicarbazones). In some embodiments, the present invention provides F₁ hybrids from elite Sorghum lines that comprises resistance to one or more ACC herbicides alone, or in conjunction with, herbicide resistance to one or more of the aforementioned herbicide groups. However, the present application is not limited to these non-ACC herbicides used, and a skilled artisan will appreciate that new non-ACC herbicides are being discovered at any given time that could be combine with herbicides that inhibit the ACC enzyme.

The application of ACC inhibitor herbicides also acts efficiently on perennial weeds, which produce shoots from rhizomes, root stocks, and other perennial organs, which are difficult to control. Here, the substances can be applied for example, by the pre-sowing method, the pre-emergence method, or the post-emergence method, for example jointly or separately. Preference is given, for example, to application by the post-emergence method, in particular to emerged harmful plants or unwanted vegetation.

Examples of weed species on which the application according to present invention act efficiently are, from amongst the monocotyledonous weed species, Avena spp., Alopecurus spp., Apera spp., Brachiaria spp., Bromus spp., Digitaria spp., Lolium spp., Echinochloa spp., Panicum spp., Phalaris spp., Poa spp., Setaria spp. and also Cyperus species from the annual group, and, among the perennial species, Agropyron, Cynodon, Imperata and Sorghum and also perennial Cyperus species.

Field crops have been classically bred through techniques that take advantage of the plants method(s) of pollination. A plant is considered “self-pollinating” if pollen from one flower can be transmitted to the same or another flower, whereas plants are considered “cross-pollinated” if the pollen has to come from a flower on a genetically different plant in order for pollination to occur.

Plants that are self-pollinated and selected over many generations become homozygous at most, if not all, of their gene loci, thereby producing a uniform population of true breeding progeny. A cross between two homozygous plants from differing backgrounds or two different homozygous lines will produce a uniform population of hybrid plants that will more than likely be heterozygous at a number of the gene loci. A cross of two plants that are each heterozygous at a number of gene loci will produce a generation of hybrid plants that are genetically different and are not uniform.

Host plant resistance is the most effective and economical approach to minimized the economic losses in crops to pests. Crop producers select cultivars, hybrids or open pollinated varieties, to plant in their fields based on host plant resistances to protect their crops some expected pests in their region. In a preferred embodiment, Sorghum farmers would select to plant Sorghum seed that can produce plants that are resistant to ACC inhibiting herbicides. The application of this trait, resistance to ACC inhibiting herbicides by Sorghum farmers facilitates the application of ACC inhibiting herbicides over their fields as a means of controlling unwanted grass species vegetation in their fields. A preferred application method would be a broadcast spray, or nonselective nor non-directed spray, over the top of the entire field that contains both Sorghum plants with resistance to ACC inhibiting herbicides and unwanted vegetation.

All publications and patents cited in this disclosure are incorporated by reference in their entirety. To the extent the material incorporated by reference contradicts or is inconsistent with this specification, the specification will supersede any such material.

The following examples are provided in order to demonstrate and further illustrate certain preferred embodiments and aspects of the present invention and are not to be construed as limiting the scope thereof.

### EXAMPLES

### Example 1: Plant Material, Isolation of Immature Embryos (IEs), Production of Sorghum Callus and Selection of FOP Herbicide Resistant Calli

Callus production from the isolation of immature embryos (IEs) was accomplished by removing immature seeds from greenhouse grown Sorghum (Inbred BTX430) panicles approximately 14 days after anthesis and surface sterilized in ethanol followed by a bleach solution and then washed in sterilized water. Immature embryos were isolated from seeds and transferred to medium with 20 IEs per plate. Cultures were grown for two weeks at (28° C.). All explants which produced somatic embryogenic calli were transferred to growth medium and cultured for an additional 4-6 days at 28° C. Explants were transferred to fresh media every three weeks until the entire callus turned into compact and friable green callus. Three weeks old freshly subcultured calli were used for the experiment.

The herbicide active ingredient molecule of ACC herbicide Assure II (Quizalofop-p- ethyl) was used for screening and selection process. For identifying the optimum concentration for screening, a kill curve experiment with concentrations ranging from 0.05 to 1.0 μM of a.i. molecules were tested. Three-week-old calli were cut into small pieces of 4-5 mm dia and 25 pieces were tested in three replications for each concentration. Concentrations used were: 0.05, 0.1, 0.2, 0.3, 0.4, 0.5 and 1 μM Quizalofop p-ethyl. As 50% of calli were killed at 0.2 uM concentration, the following selection criteria was followed for entire screening process. Selection process was started with 0.05 um and doubled the concentration for every 3 weeks and the calli surviving on 1.0 uM was considered as resistant calli.

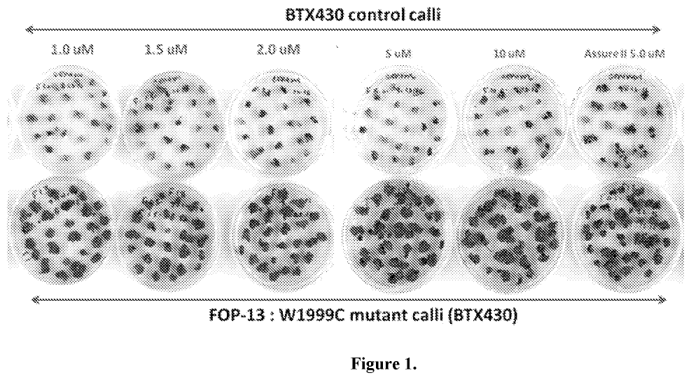

For the chemical mutagenesis, two chemical mutagens ethyl methanesulfonate (EMS) (0.25 and 0.5%) and sodium azide (0.5 and 1.0 uM) were used. These mutagens at specified concentration were directly added into the culture media. The calli pieces were first cultured on the media containing the mutagens for 6 and 12 hrs and then transferred to the above-mentioned selection procedure. A total of 16 experiments were conducted with varying amount of calli pieces. After 21 to 24 weeks of selection, the surviving calli were considered as herbicide resistant calli. The surviving resistant calli were further tested on 1, 2, 5, 10, 20, 50 and 100 μM concentration of ai molecule Quizalofop-p-ethyl and the herbicide Assure II (FIG. 1).

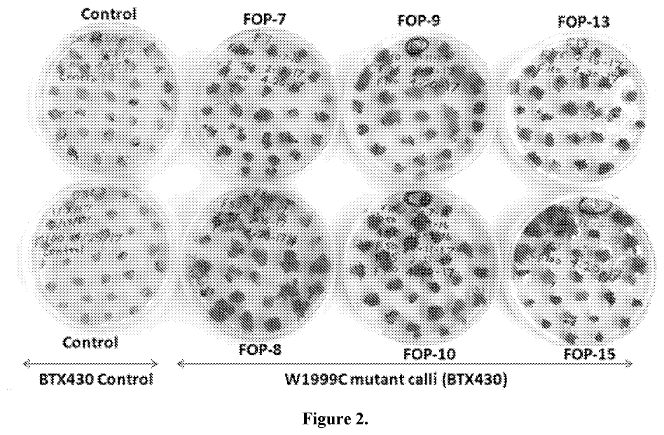

A total of 19 resistant calli (FP-1 to FP-19) were obtained from 16 experiments. Out of 19 resistant calli, 3 resistant calli (FP-8, FP-11, and FP-12) were obtained from chemical mutagenesis. The resistant calli FP-7 to FP16 were showed high level of resistant to both Quizalofop-p-ethyl a.i. molecules and the herbicide Assure II and survived up to 100 concentration (FIG. 2).

### Example 2: Plant Regeneration, DNA Isolation and Sequence Analysis

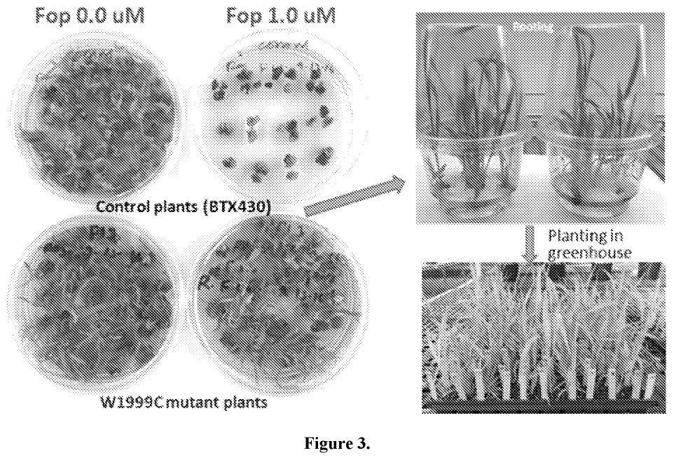

The resistant calli were transferred onto regeneration medium either supplemented with 0 or 1.0 uM of Quizalofop-p-ethyl. Well-developed shoots were transferred onto hormone free rooting medium containing 0 or, 1, 2.5 and 5 μM Quizalofop-p-ethyl. Rooted plants were transferred into the greenhouse and established into a whole plant (FIG. 3). Several hundred plants were regenerated from these resistant calli and established as whole plants in the greenhouse. The plants regenerated from resistant calli (FP-8, FP-11 and FP-12) through chemical mutagenesis of Sorghum calli, showed dwarf plant phenotype and male sterility.

For sequencing experiments, DNA was isolated from the both resistant calli and the regenerated plants. The entire 1863 bp Carboxyl Domain (CT) region was PCR amplified using the four pairs of the overlapping primers (Table 1).

TABLE 1
Primers used to amplify the CT domain region
of sorghum ACC gene.

| | | Product | SEQ |
|---|---|---|---|
| PCR | | Size | ID |
| primers | DNA sequences | (bp) | NO: |
| 1F | 5′GCAACTCTGGTGCTAGGATTGGCA3′ | 553 | 11 |
| 1R | 5′GAACATAGCTGAGCCACCTCAATAT | | 12 |
| | ATT3′ | | |
| 2F | 5′GGTGGTCCTAAGATCATGGCGACC3′ | 790 | 13 |
| 2R | 5′AGTCTTGGAGTTCCTCTGACCTGAAC3′ | | 14 |
| 3F | 5′CAGCTTGATTCCCATGAGCGATC3′ | 406 | 15 |
| 3R | 5′CCATACAGTCTTGGAGTTCCTCTGA3′ | | 16 |
| 4F | 5′GAGTGTTATGCTGAGAGGACTGCCAA3′ | 711 | 17 |
| 4R | 5′ACCAAGGACCTTCTTGACTTCCTG3′ | | 18 |

Two step PCR reaction was performed using New England Biolabs multiplex PCR master mix kit, in a 50 μl reaction mixture containing 2 μl of total DNA (100 ng), 10 μl × PCR buffer, 2 μl of primer mix (5 μm/μl of each forward and reverse primer), 0.7 μl of 10 μM dNTPs, 0.5 μl of Phusion high fidelity DNA polymerase and 34.8 μl of sterile distilled water. The CT domain sequences were amplified with the following temperature conditions: Pre incubation at 98° C. for 3 min, then 35 cycles of denaturation at 98° C. for 20 sec, annealing and extension at 72° C. for 1.2 min, followed by a final extension at 72° C. for 10 min. For sequencing reaction, the entire 1.863 kb CT domain region was PCR amplified using 1F and 4R primers. The PCR products were purified using QIAGEN® (Qiagen GmbH, Hilden, Germany) columns and sequenced at University of Chicago Sequencing Facility using the combination of primers mentioned above. The sequencing results were aligned and compared using Vector NTI program.

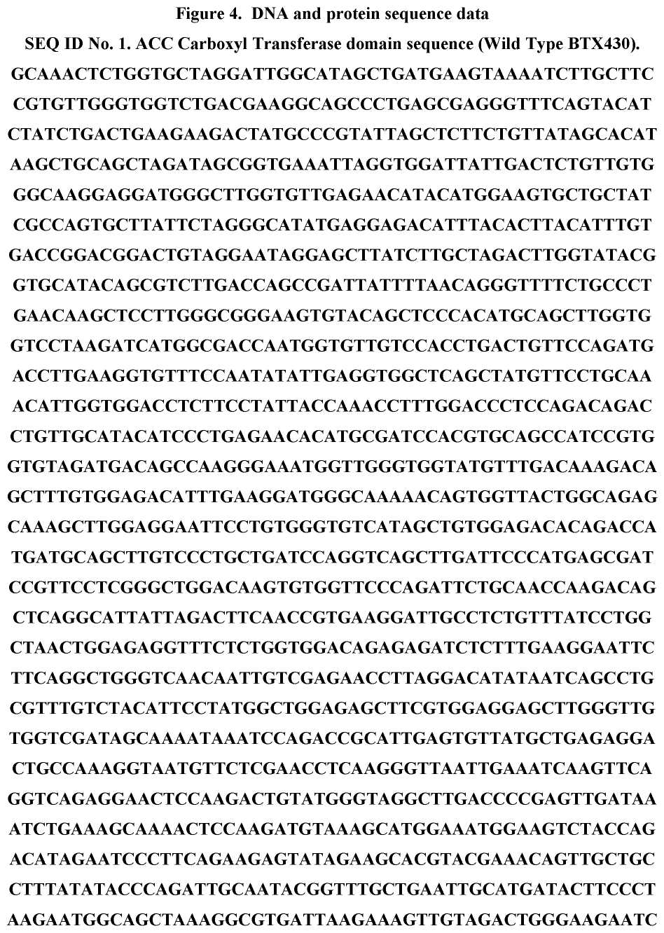

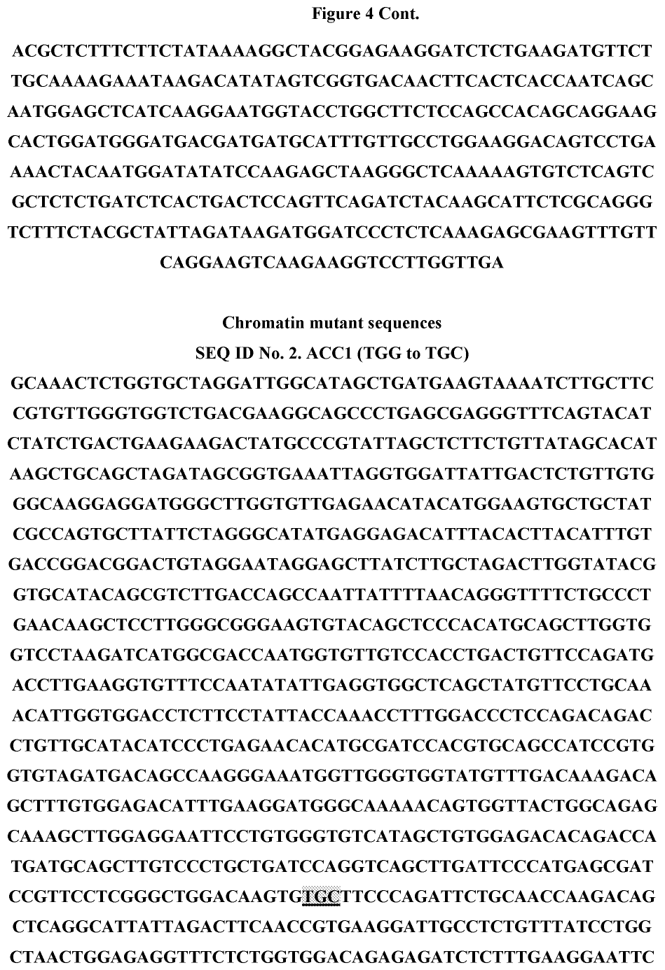

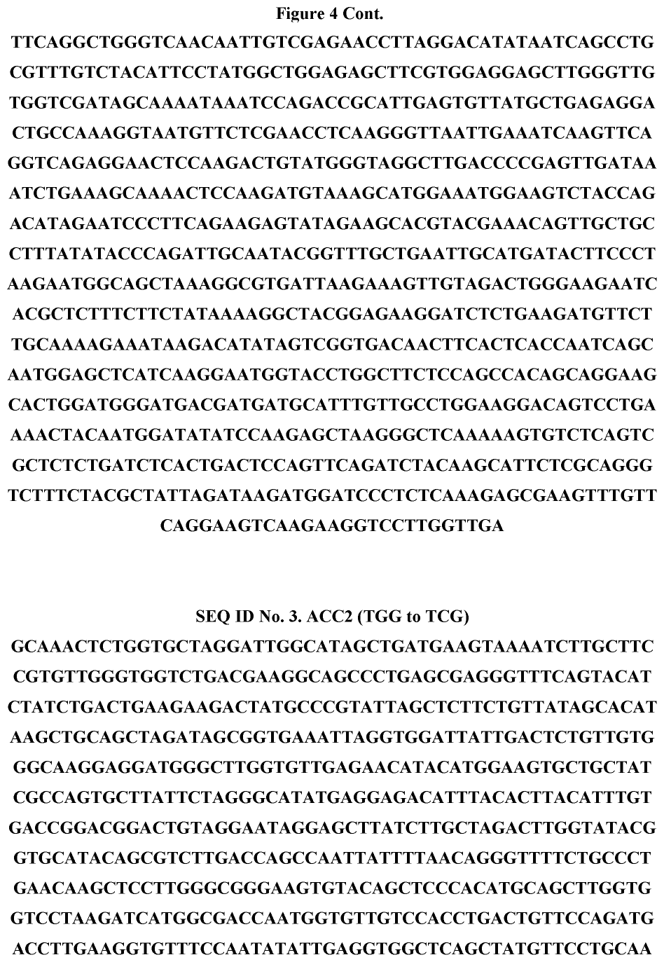

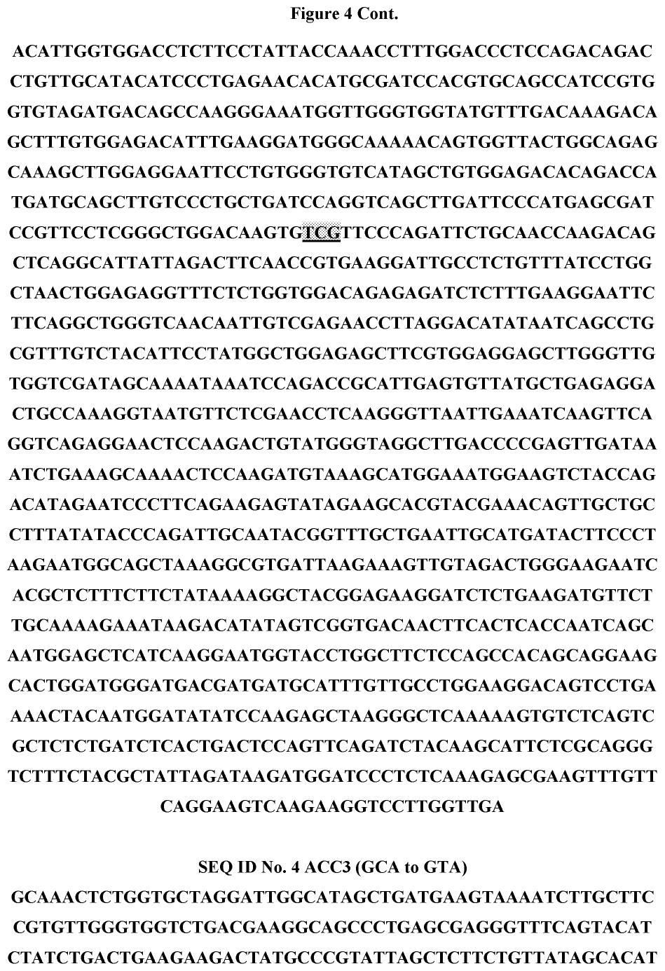

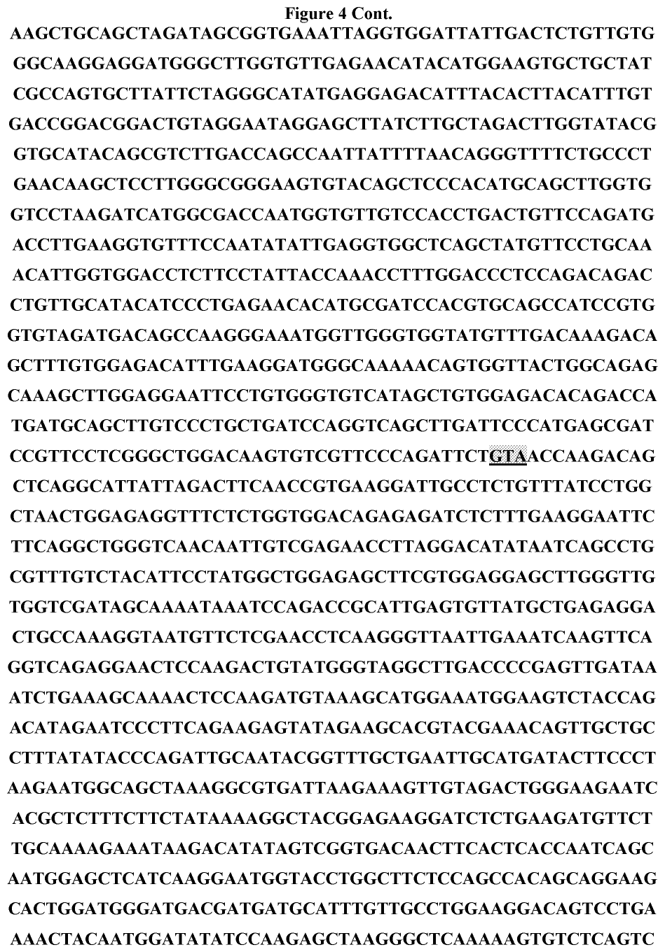

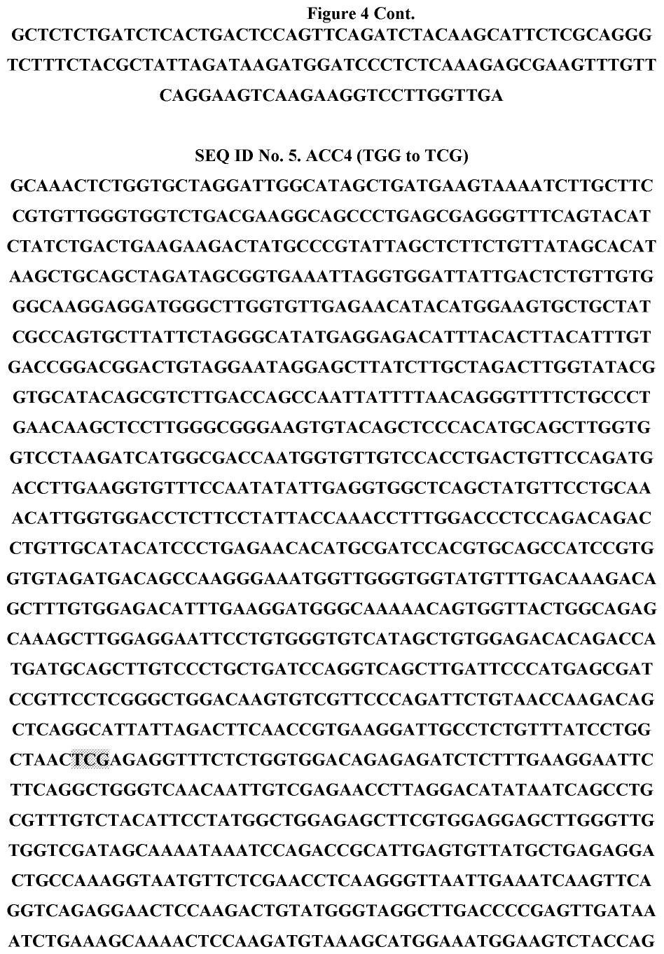

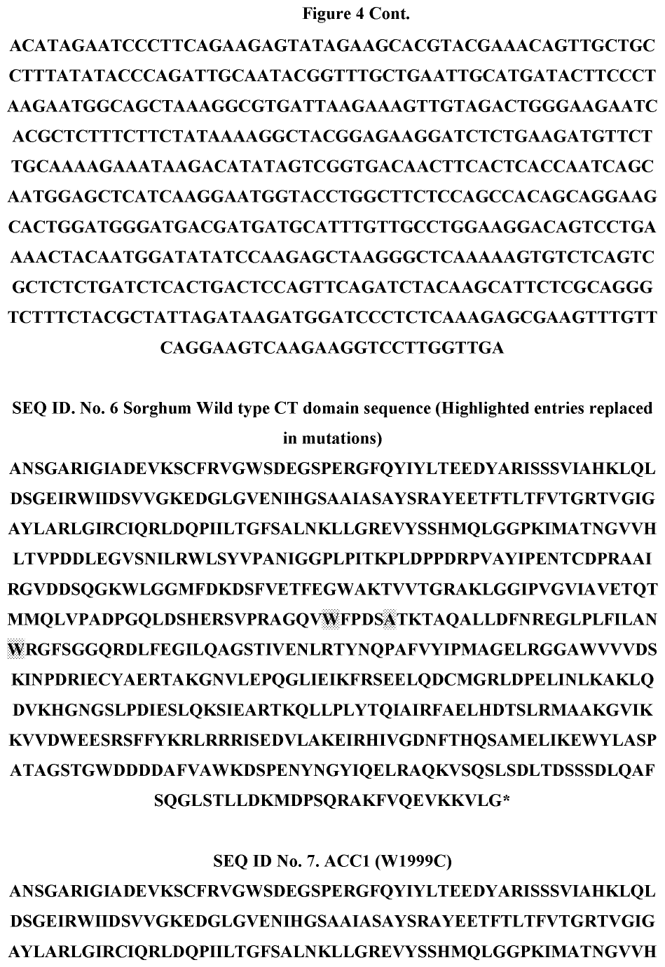

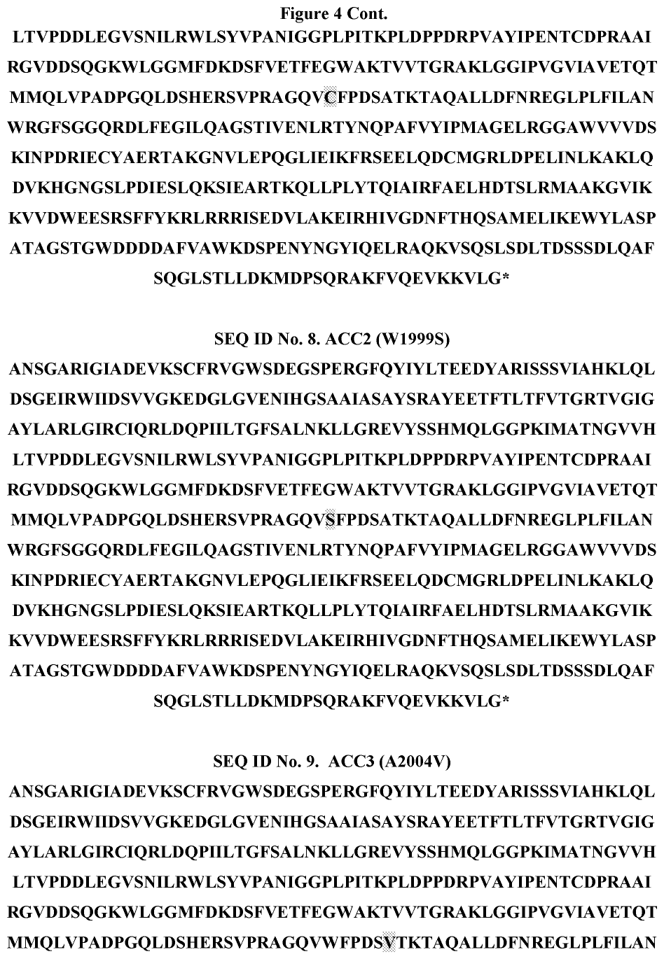

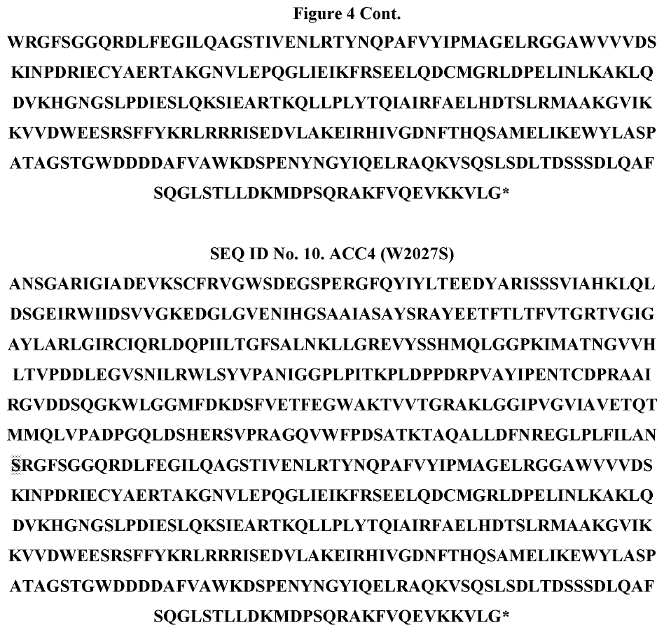

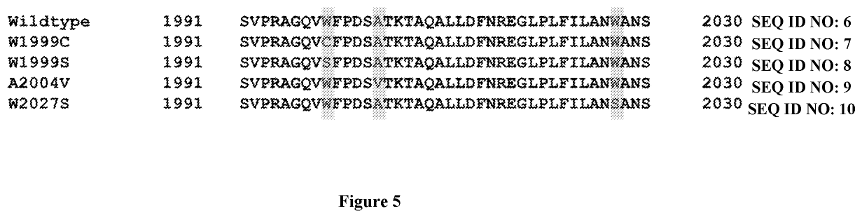

The DNA and amino acid sequences of wild type genotype (BTX430) were shown in SEQ ID NO: 1 and 6, respectively (FIG. 4). The sequencing result showed that the DNA isolated from FP-7, FP-8, FP-9, FP-10, FP-13, FP-14, FP-15 and, FP-16 resistant calli or plants have mutation of TGG to TGC (SEQ ID NO: 2) that leads to replacing the amino acid Tryptophan with Cysteine at the amino acid codon position aligning with 1999Trp of the blackgrass weed A. myosuroides (W1999C) ACC protein (SEQ ID NO: 7). DNA from FP-11 and FP-12 resistant calli or plants have mutation of TGG to TCG (SEQ ID NO: 3) that leads to replacing Tryptophan with Serine at codon position W1999S, (SEQ ID NO: 8). DNA from FP-17 has mutation of GCA to GTA (SEQ ID NO: 4) that leads to replacing Alanine with Valine at codon position A2004V (SEQ ID NO: 9). DNA from FP-4, FP-5, FP-6, FP-18 and FP-19 have mutation of TGG to TCG (SEQ ID NO: 5) that leads to replacing Tryptophan with Serine at codon position W2027S, (SEQ ID NO: 10). Part of the protein mutation sequences were aligned, compared and shown in FIG. 5. All the SNP mutations except FP-8 and FP-11, showed heterozygous conditions for ACC mutation while FP-8 and FP-11 were homozygous mutants.

### Example 3: Screening of Herbicide Resistance Under Greenhouse Conditions with Quizalofop Herbicide Assure II

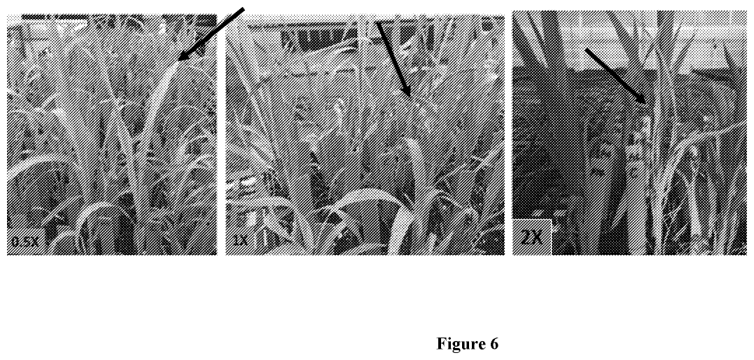

ACC herbicide resistance was tested under greenhouse conditions at different growth stages. At first, two- to three-week-old greenhouse grown F₀ plants generated from resistant calli were screened along with control plants for herbicide resistance by spraying with 2.5, 5.0, or 10 oz of Assure II herbicide per acre. These rates represent 0.5, 1.0, and 2.0 times (×), respectively, the labeled rate for control of wild Sorghum or shattercane. Herbicide were applied at the field application rate of 15 gallons of herbicide and water mixture per acre either using small hand sprayer or CO₂ pressurized sprayer. Plants were rated 15 days after herbicide application as alive or dead. Herbicide application results showed that plants regenerated from FP-4 to and FP-19 were resistant to either 0.5×, 1× or 2× rate of herbicide application. However, plants from FP-7 to FP-16 were highly resistant to herbicide application up to a 2× rate (FIG. 6) while FP-4, FP-5, FP6, FP18 and FP19 showed moderate level of resistance up to 1X rate and were dead at 2X rate. FP-1, FP-2 and FP-3 were dead after spraying with 1× rate. All the control plants were dead even at 0.5× rate. The surviving plants were transferred into bigger pots for further analysis and allowed to set seed.

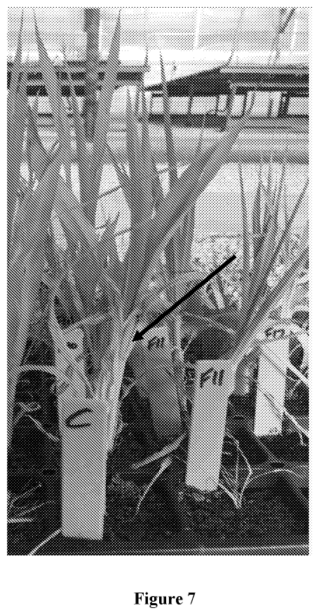

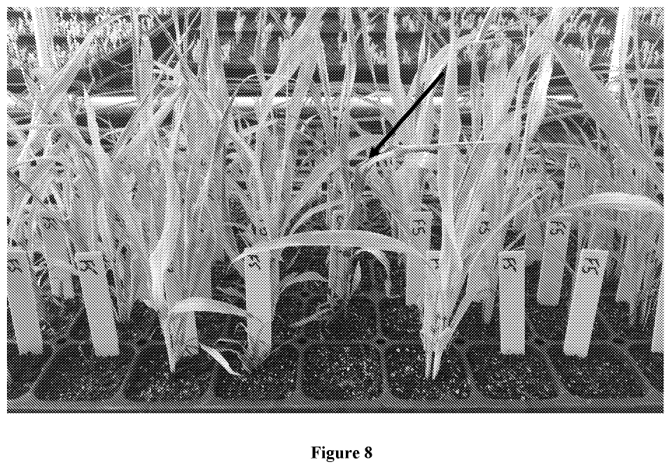

ACC herbicide resistance was tested under greenhouse conditions at different growth stages. At first, two- to three-week-old greenhouse grown F₀ plants generated from resistant calli were screened along with control plants for herbicide resistance by spraying with 2.5, 5.0, or 10 oz of Assure II herbicide per acre. These rates represent 0.5, 1.0, and 2.0 times (×), respectively, the labeled rate for control of wild Sorghum or shattercane. Herbicide were applied at the field application rate of 15 gallons of herbicide and water mixture per acre either using small hand sprayer or CO₂ pressurized sprayer. Plants were rated 15 days after herbicide application as alive or dead. Herbicide application results showed that plants regenerated from FP-4 to and FP-19 were resistant to either 0.5×, 1× or 2× rate of herbicide application. However, plants from FP-7 to FP-16 were highly resistant to herbicide application up to a 2× rate (FIG. 6 and FIG. 7) while FP-4, FP-5, FP6, FP18 and FP19 showed moderate level of resistance up to 1× rate and were dead at 2× rate (FIG. 8). FP-1, FP-2 and FP-3 were dead after spraying with 1× rate. All the control plants were dead even at 0.5× rate. The surviving plants were transferred into bigger pots for further analysis and allowed to set seed.

Mature F₀ Sorghum plants were also screened for herbicide resistance by spraying higher field rate of herbicide Assure II at 8.0, 16.0 or 32 oz of Assure II herbicide per acre. These rates represent 1.0, 2.0 and 4.0 times (×), respectively, the labeled rate for control of wild Sorghum or shattercane. Six to eight weeks old control (BTX430) and W1999C mutant plants were sprayed with 1×, 2× and 4× rates using a small hand sprayer at the application rate of 15 gallons of herbicide and water per acre. Each pot containing 3 to 4 plants were sprayed with 75 to 100 ml of herbicide solution or until runoff. Plants were graded 3 weeks after application. The herbicide application results showed that W1999C mutant Sorghum plants were unaffected by herbicide application even at 4× rate whereas the control plants were killed at the 1× rate (FIG. 9). The herbicide resistant plants grew into normal plants and set seed.

The resistant plants were grown to maturity and set seed either by self or cross pollinating with pollen from other Sorghum inbred lines. The harvested F₁ seed was planted in the greenhouse and inheritance of herbicide resistance was also demonstrated in two-week-old young plants by spraying Assure II at 2× rate (FIG. 10). Good seed set was noticed in most of the plants (FIG. 11) and seed was produced for all mutants except A2004V.

### Example 4: Development of KASP DNA Markers for ACC Gene Mutations

Four SNP (Single Nucleotide Polymorphisms) mutations (TGG to TGC at codon position 1999, TGG to TCG at codon position 1999, GCA to GTA at codon position 2004 and TGG to TCG at codon position 2027) in the CT domain of the ACC gene of wild-type Sorghum (SEQ ID NO: 1) are responsible for the herbicide resistance in BTX430 Sorghum. The KASP (Kompetitive Allele Specific PCR) DNA markers were developed for all SNPs except mutation at codon 2004 using the following primers (Table 2) and the procedures described at the LGC website describing genotyping chemistry using KASP.

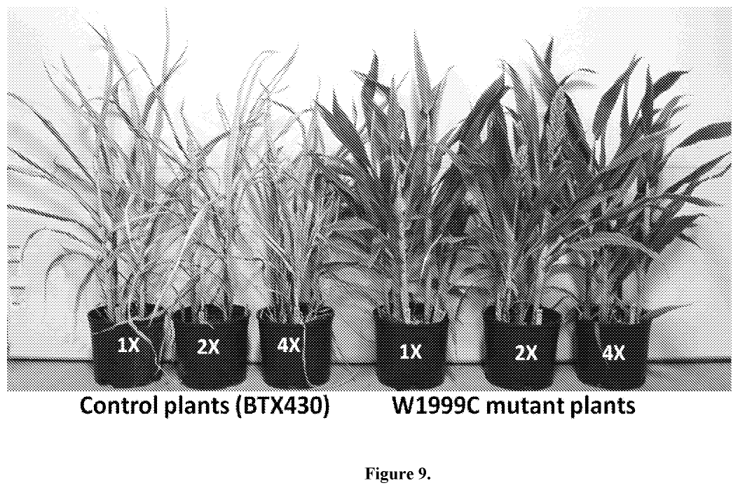

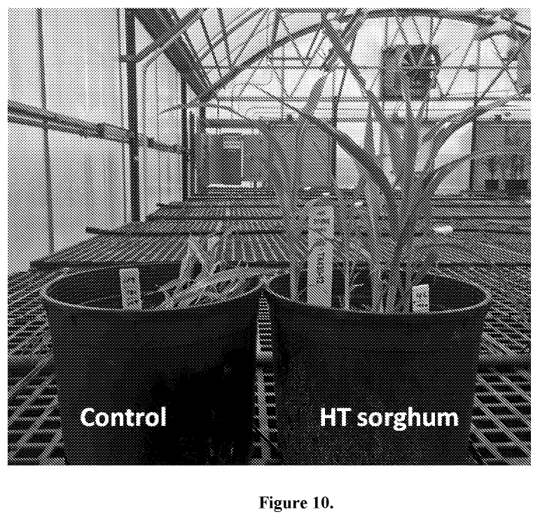

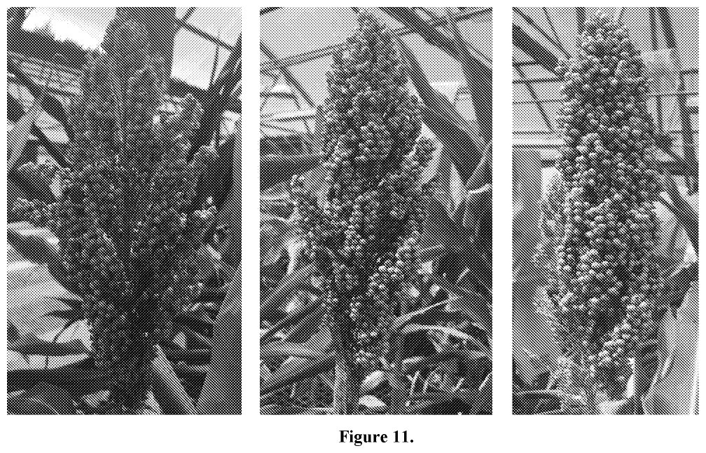

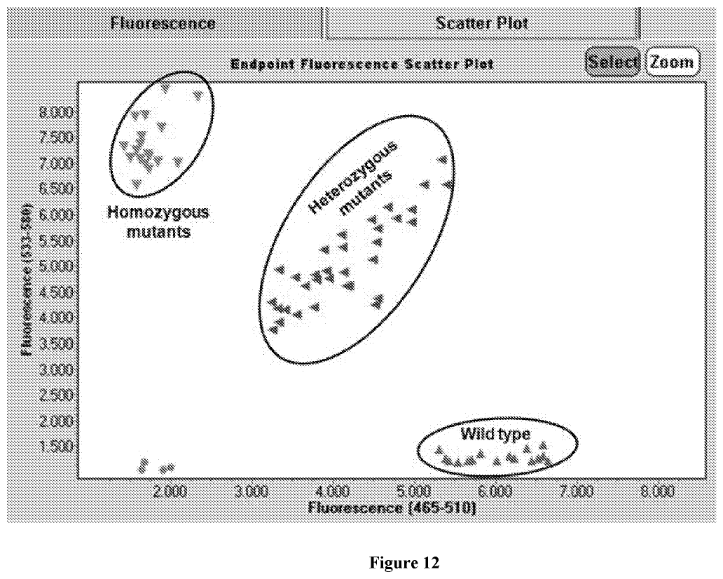

KASP was performed by a real time PCR allelic discrimination assay using a Roche Light Cycler 480 II thermocycler (Roche Diagnostics GmbH, Roche Applied Science, 68298 Mannheim, Germany). A PCR reaction mix was prepared in a final volume of 10 μl reaction comprising of 5 μl of 2× KASP master mix (LGC), 0.14 μl primer mix (X+Y+C), and 25 ng of genomic DNA with DNase-free water to make up the final volume. PCR amplification conditions: one initial denaturation cycle at 94° C. for 15 min, followed by 10 cycles of initial amplification at 94° C. for 20 seconds and annealing/extension at 61-55° C. for 1 min (dropping 0.6° C. per cycle). Then 30 cycles of amplification program at 94° C. for 20 seconds and 55° C. for 1 min. The result of the allelic discrimination was determined by Endpoint detection of fluorescence following Roche LightCycler@480 Instrument Operator's Manual. The KASP assay differentiated the homozygous and heterozygous nature of the herbicide resistance and separated the wild types in segregating populations (FIG. 12). The developed KASP markers were used to genotype the resistant calli, F₀, F₁ and F₂ segregating population.

TABLE 2
Primers used in KASP assay for detecting W1999C,
W1999S and W2027S mutations.

| | | | SEQ |
|---|---|---|---|
| | | | ID |
| SNPs | PCR primers | DNA sequences | NO: |
| TGG to | X (HAX dye) | 5′GGGCTGGACAAGTGTGG3′ | 19 |
| TGC at | Y (FAM dye) | 5′GGGCTGGACAAGTGTGC3′ | 20 |
| 1999 | C | 5′CTGAGCTGTCTTGGTTGCAG3′ | 21 |
| TGG to | X (HAX dye) | 5′TTGCAGAATCTGGGAACC3′ | 22 |
| TCG | Y (FAM dye) | 5′TTGCAGAATCTGGGAACG3′ | 23 |
| at 1999 | C | 5′GGTCAGCTTGATTCCCATGA3′ | 24 |
| TGG to | X (HAX dye) | 5′GTCCACCAGAGAAACCTCTCC3′ | 25 |
| TGC | Y (FAM dye) | 5′GTCCACCAGAGAAACCTCTCG3′ | 26 |
| at 2027 | C | 5′CGTGAAGGATTGCCTCTGTT3′ | 27 |

X: Wild type allele;
Y: Mutant allele;
C: Reverse primer

### Example 5. Correlating the Herbicide Resistance with W1999C Mutation in F₂ Segregating and Homozygous Lines of BTX430.

The BTX430 Sorghum plants with W1999C mutation showing high level of herbicide resistance were self-pollinated and also cross-pollinated with tissue culture derived wild type BTX430 to produce subsequent generations. Several lines were grown in the greenhouse to maturity to set seed. Except few plants, most of the F₀ plants were fertile and shed pollen. Plants regenerated from FP-7, FP-9, FP-10, FP-13, FP-14, FP-15 and FP-16 resistant calli all produced F₁ seed in the greenhouse. Twenty F₁ seed for each line were planted in the greenhouse and screened with the application of herbicide at the 2× (16.0 oz/acre) herbicide rate. A total of 54 surviving F₁ plants (F₁-P1 to F₁-P54) comprising of 22 homozygous and 32 heterozygous plants were transferred to bigger pots and grown to set seed by self-pollination. Five plants from each group were also sequenced to confirm the mutation and the zygosity. After harvesting F₂ seed upon maturity, four lines from each group (F₁-P8, F₁-P12, F₁₆-P16 and F₁-29 for homozygous) and (F₁-P3, F₁-P20, F₁-P30 and F₁-P50) were selected for genotyping and to know the inheritance of herbicide tolerance. Fifty F2 plants were genotyped and tested for herbicide resistance by spraying Assure II at the 2× rate (16 oz/acre) at 2 weeks after planting using CO₂ pressurized sprayer with spray volume of 15 gallons of herbicide and water mixture per acre. Wild type BTX430 plants generated through tissue culture was used as control. Two weeks after spraying, the plants were rated for resistance as alive or dead. Genotyping data of F₂ plants showed that all the plants from F₁ homozygous lines were homozygous (TGC) for herbicide resistance. Except 4 plants, all the F₂ homozygous plants were alive and exhibited high level of herbicide resistance (Table 3). As expected, the plants from F₁ heterozygous lines showed three kinds of genotypes, TGC (homozygous mutant); TGG/C (heterozygous mutant for herbicide resistance) and TGG (wild type) confirming the Mendelian segregation ratio of 1:2:1 with the Chi square value of 0.88, 0.24, 0.53 and 0.22 which is less than the table value of 2.706. This means the segregation ratios are in goodness of fit with no significant difference between expected and observed value. Herbicide application resulted the death of 87% of wild type plants. The remaining wild type plants did not die, but developed dead heart symptoms. Some of the small heterozygous plants showed slight yellowing phenotypes, but later recovered. The presence of W1999C mutation in the F2 segregating population either in homozygous (TGC) or heterozygous (TGG/C) conditions clearly correlated with herbicide resistance with correlation co efficient values of 0.937, 0.841, 0.783, and 0.715 for F₂-P3, F₂-P20, F₂-P30 and F₂-P50, respectively.

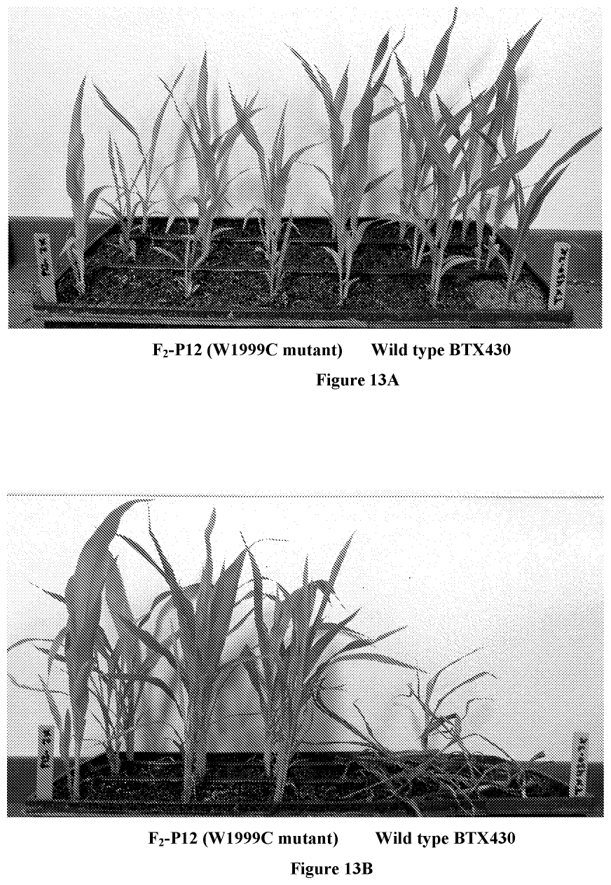

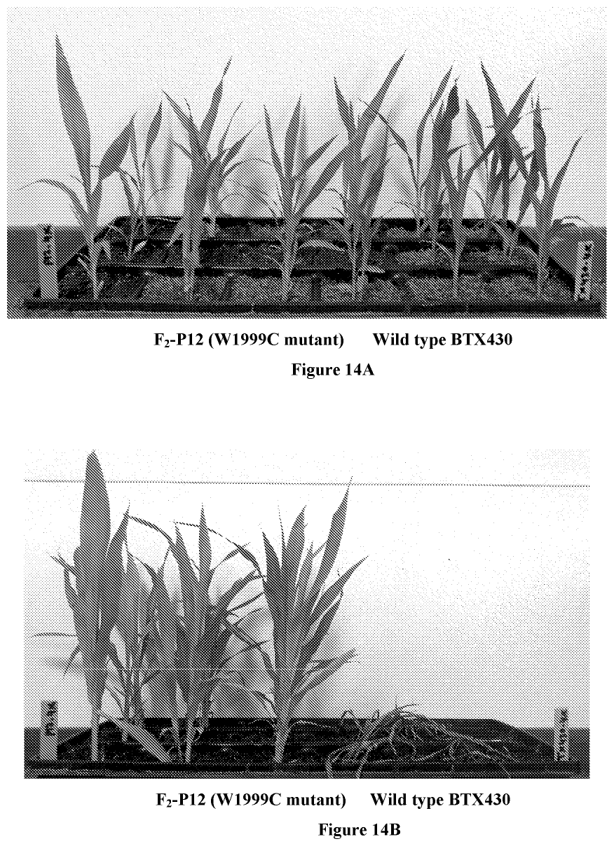

In addition, eighteen plants from the F₂ homozygous W1999C mutant line (P12) along with control BTX430 plants were tested for herbicide resistance by spraying with 2× and 4× rates (16 and 32 oz/acre) using a CO₂ pressurized sprayer with a spray volume of 15 gallons of herbicide and water mixture per acre. Plants were rated 14 days after herbicide application as alive and dead. The herbicide application results showed that all the homozygous F₂ plants survived both at 2× (FIGS. 13A and 13B) and 4× (FIGS. 14A and 14B) rates, and all the control BTX430 plants were killed at both application rates.

TABLE 3
Genotype and phenotype data of selected F₂ lines.

| | | No. of | | | | | |
|---|---|---|---|---|---|---|---|
| | F₁ genotype for | plants | F₂ genotype | | | F₂ phenotype | |
| Lines | W1999C mutant | tested | TGC | TG(G/C) | TGG | Alive | Dead |
| F₂-P8 | Homozygous | 50 | 0 | 0 | 50 | 49 | 1 |
| F₂-P12 | Homozygous | 50 | 0 | 0 | 50 | 50 | 0 |
| F₂-P16 | Homozygous | 50 | 0 | 0 | 50 | 50 | 0 |
| F₂-P29 | Homozygous | 50 | 0 | 0 | 50 | 47 | 3 |
| F₂-P3 | Heterozygous | 50 | 12 | 28 | 10 | 41 | 9 |
| F₂-P20 | Heterozygous | 50 | 14 | 24 | 12 | 37 | 13 |
| F₂-P30 | Heterozygous | 34 | 7 | 17 | 10 | 25 | 9 |
| F₂-50 | Heterozygous | 41 | 11 | 21 | 9 | 32 | 9 |
| BTX430 | Wild type | 50 | 0 | 0 | 50 | 0 | 50 |

### Example 6. Introgression of Herbicide Resistant Trait into Elite Germplasms Through Recurrent Back Crossing

Herbicide resistance was introgressed into 33 inbred lines which includes both R and B lines for Sorghum hybrid production. For backcrossing, the elite inbred lines were emasculated and crossed with the herbicide resistant BTX430-CHR-ACC plants as male parent. After pollination, plants were allowed to set seed. F₁ seed was harvested and used for recurrent backcrossing with their respective parents. For this purpose, 36 seed from each cross was planted and screened for herbicide resistant plants by spraying with Assure II at the 2× (16 oz/acre) rate as described before. The higher rate of Assure II (8 oz/acre) was used as the basis for screening seedling plants. All the surviving plants were genotyped using the KASP DNA marker for W1999C mutant to eliminate any “escapes” or wild types. The segregating heterozygous F₁ herbicide resistant plants were used as the male parent in the back-crossing process to produce F₂ generations.

### CITED REFERENCES

1. Armstrong, J. M. et al., 2015. Sugarcane Aphid (Hemiptera:Aphididae): Host Range and Sorghum Resistance Including Cross-Resistance from Greenbug Sources. J. of Econ Entomol. 108: 576-582.
2. Ausubel, F. M. et al., 1989. Current Protocols in Molecular Biology. Vol. 1 and 2. Wiley & Sons, Inc., Media, Pa.
3. Blochlinger, K and Diggelmann, H., 1984. Hygromycin B phosphotransferase as a selectable marker for DNA transfer experiments. Mol. Cell. Biol. 4:2929-2931.
4. Bourouis, M. and Jary, B., 1983: EMBO J. 2:1099-1104.
5. Carlson, S. R. et al., 2007. Meiotic transmission of an in vitro-assembled autonomous maize minichromosome. PLoS Genet. 3:1965-1974.
6. Casas, A. M. et al., 1993. Transgenic Sorghum plants via microprojectile bombardment. Proc. Natl. Acad. Sci. USA 90:11212-11216.
7. Chao, W. S. et al., 1999. Leucine Aminopeptidase RNAs, Proteins, and Activities Increase in Response to Water Deficit, Salinity, and the Wound Signals Systemin, Methyl Jasmonate, and Abscisic Acid1. Plant Physiol. 120:979-992.
8. Christoffers, M. J. et al., 2002. An Isoleucine to Leucine Mutation in AcetylCoA Carboxylase Confers Herbicide Wild Oat. Genome 45:1049-1056.
9. Crossway, A. et al., 1985. Integration of foreign DNA following microinjection of tobacco mesophyll protoplasts. Mol. Gen. Genet. 202:179-185.
10. Deyle, C., 2002. An isoleucine-leucine substitution in chloroplastic acetyl-CoA carboxylase from green foxtail (Setaria viridis L. Beauv.) is responsible for resistance to the cyclohexanedione herbicide sethoxydim. Planta. 214:421-427.
11. Deyle, C. et al., 2005. Molecular Bases for Sensitivity to Acetyl-Coenzyme A Carboxylase Inhibitors in Black-Grass. Plant Physiol. 137:794-806.
12. Deyle, C. et al., 2002. PCR-based detection of resistance to acetyl-CoA carboxylase- inhibiting herbicides in black-grass. Pest Management Science 58:474-478.
13. Deyle et al., 2002. SNP markers for black-grass (Alopecurus myosuroides Huds.) genotypes resistant to acetyl CoA-carboxylase inhibiting herbicides. Theor. Appl. Genet. 104:1114-1120.
14. Evans D. A. et al., 1983. Handbook of Plant Cell Cultures, vol. 1, 1983, MACMILLAN PUBLISHING CO.
15. Fehr, W. R., 1993. Principles of Cultivar Development: Theory and Technique. Macmillan Publishing Company. p. 536.
16. Fraley, R. T. et al., 1982. Proc. Natl. Acad. Sci. USA 79:1859-1863.
17. Fraley, R. T. et al., 1983. Expression of bacterial genes in plant cells. Proc. Natl. Acad. Sci. USA 80:4803-4807.
18. Fromm, M. et al., 1985. Expression of genes transferred into monocot and dicot plant cells by electroporation. Proc. Natl acad. Sci. USA. 82: 5824-5828.
19. Guerineau et al., 1991. Mol. Gen. Genet. 262:141-14.
20. Gurel, S. et al., 2009. Efficient, reproducible Agrobacterium-mediated transformation of Sorghum using heat treatment of immature embryos. Plant Cell Rep. 28:429-444.
21. Herbert et al., 1996. Kinetic studies on two isoforms of acetyl-CoA carboxylase from maize leaves. Biochem. J. 318:997-1006.
22. Herrera-Estrella, 1983. Expression of chimaeric genes transferred into plant cells using a Ti-plasmid-derived vector. Nature. 303: 209-213
23. Hinchee et al., 1988. Production of Transgenic Soybean Plants Using Agrobacterium-Mediated DNA Transfer. Nature Biotechnol. 6: 915-922.
24. Ishida et al., 1996, High efficiency transformation of maize (Zea mays L.) mediated by Agrobacterium tumefaciens. Nature Biotechnol. 14:745-750.
25. Joshi, 1987. An inspection of the domain between putative TATA box and translation start site in 79 plant genes. Nucleic Acids Res. 15: 6643-6653.
26. Kalderon et al., 1984. A short amino acid sequence able to specify nuclear location. Cell 39: 499-509.
27. Krens et al., 1982. In vitro transformation of plant protoplasts with Ti-plasmid DNA. Nature 296:72-74.
28. Luehrsen and Walbot, 1991. Intron enhancement of gene expression and the splicing efficiency of introns in maize cells. Mol. Gen. Genet. 225: 81-93.
29. Messing and Vierra. 1982, The pUC plasmids, an M13mp7-derived system for insertion mutagenesis and sequencing with synthetic universal primers. Gene. 19:259-268.
30. Nikolau, B. J. et al., 2003. Plant Biotin-Containing Carboxylases. Archives of Biochemistry and Biophysics 414: 211-222.
31. Odell, J. T. et al., 1985. Identification of DNA sequences required for activity of a plant promoter: the CaMV 35S promoter. Nature 313:810-812.
32. Paszkowski, J. et al., 1984. Direct gene transfer to plants. EMBO J. 3:2717-2722.
33. Proudfoot, N., 1991. Poly(A) signals. Cell. 64: 671-674.
34. Rendina et al., 1990. Inhibition of Acetyl Co enzyme carboxylass by two classes of grass selective herbicides. J. Agric. Food Chem. 38:1282-87.
35. Riggs and Bates, 1986. Stable transformation of tobacco by electroporation: evidence for plasmid concatenation. Proc. Natl. Acad. Sci. USA 83:5602-5606.
36. Rosenberg, A. H. et al., 1987. Vectors for selective expression of cloned DNAs by T7 RNA polymerase. Gene 56:125-135.
37. Sambrook, et al., 1989. Molecular Cloning, A Laboratory Manual, COLD SPRING HARBOR PRESS.
38. Sanfacon et al., 1991. A dissection of the cauliflower mosiac virus polyadenylation signal. Genes Dev. 5:141-149.
39. Schell 1987. Transgenic plants as tools to study the molecular organization of plant genes. Science 237:1176-1183.
40. Sleper, D. A and Poehlman, J. M., 2006. Breeding Field Crops, Fifth Edition, Blackwell Publishing, p. 424.
41. Staub, J. M. and Maliga, P., 1992. Long regions of homologous DNA are incorporated into the tobacco plastid genome by transformation. Plant Cell. 4:39-45.
42. Staub, J. M. and Maliga, P., 1993. Accumulation of D1 polypeptide in tobacco plastids is regulated via the untranslated region of the psbA mRNA. EMBO J. 12:601-606.
43. Svab et al., 1990. Stable transformation of plastids in higher plants. Proc. Natl. Acad. Sci. USA 87:8526-8530.
44. Svab, Z, and Maliga, P., 1993. High-frequency plastid transformation in tobacco by selection for a chimeric aadA gene. Proc. Natl. Acad. Scil. 90:913.
45. Vasil, 1984. Cell Culture and Somatic Cell Genetics of Plants, vol. I, III, 1984, Acad. Press.
46. White et al., 1990. A cassette containing the bar gene of Streptomyces hygroscopicus: a selectable marker for plant transformation. Nucleic Acids Res. 18:1062.
47. Zagnitko et al., 2001. An isoleucine/leucine residue in the carboxyltransferase domain of acetyl-CoA carboxylase is critical for interaction with aryloxphenoxypropionate and cyclohexanedione inhibitors, Proc. Natl. Acad. Sci. USA 98:6617-6622.
48. Zhang, X. Q. and Devine, M.D., 2000. A possible point mutation of plastidic ACCase gene conferring resistance to sethoxydim in green foxtail (Setaria viridis). Abstr. Weed Sci. Soc. Am. 2000 40:33.

### CITED PATENTS

1. U.S. Pat. No. 9,617,530. Tuinstra and Al-Khatib, 2017. Acetyl-CoA carboxylase herbicide resistant Sorghum.
2. WO2008073800AS. Tuinstra and Al-Khatib. 2008. Acetolactate synthase herbicide resistant Sorghum.
3. U.S. Pat. No. 5,187,267. 1993. Calgene, Inc. Plant proteins, promoters, coding sequences and use.
4. U.S. Pat. No. 5,057,422. 1991. Mogen International N.V. Recombinant dna: transformed microorganisms, plant cells and plants: a process for introducing an inducible property in plants, and a process for producing a polypeptide or protein by means of plants or plant cells.
5. U.S. Pat. No. 5,981,839. 1999. Calgene, LLC. Methods and compositions for regulated transcription and expression of heterologous genes.
6. U.S. Pat. No. 6,051,757. 2000. Washington University. Regeneration of plants containing genetically engineered T-DNA.
7. U.S. Pat. No. 5,981,840. 1999. Pioneer Hi-Bred International, Inc. Methods for Agrobacterium-mediated transformation.
8. U.S. Pat. No. 5,824,877. 1998. Monsanto Company Method for soybean transformation and regeneration.
9. U.S. Pat. No. 4,940,838. 1990. Schilperoort Robbert A. Process for the incorporation of foreign dna into the genome of dicotyledonous plants.
10. WO 84/02913. 1997. Glyphosate-tolerant 5-enolpyruvylshikimate-3-phosphate synthases.
11. U.S. Pat. No. 5,501,967. 1996. Mogen International, N.V./Rijksuniversiteit Te Leiden. Process for the site-directed integration of DNA into the genome of plants.
12. U.S. Pat. No. 5,846,795. 1998. Mycogen Plant Science, Inc. RNA transformation vector.
13. U.S. Pat. No. 5,500,360. 1996. Mycogen Plant Science, Inc. RNA transformation vector.
14. U.S. Pat. No. 5,173,410. 1992. Lubrizol Genetics Inc. Transfer vector.
15. U.S. Pat. No. 5,965,794. 1999. Biosource Technologies, Inc. Viral amplification of recombinant messenger RNA in transgenic plants.
16. U.S. Pat. No. 5,451,513. 1995. The State University of New Jersey RutgersMethod for stably transforming plastids of multicellular plants.
17. U.S. Pat. No. 5,545,817. 1996. Calgene, Inc. Enhanced expression in a plant plastid.
18. U.S. Pat. No. 6,369,298. 2002. Pioneer Hi-Bred International, Inc. Agrobacterium mediated transformation of Sorghum.
19. U.S. Pat. No. 4,945,050. 1990. Cornell Research Foundation, Inc. Method for transporting substances into living cells and tissues and apparatus therefor.
20. WO1993007278A1. 1993. Ciba Geigy Ag. Synthetic dna sequence having enhanced insecticidal activity in maize.

We claim:

1. A plant of elite Sorghum line BTX430-CHR-ACC1 with ACC inhibitor herbicide resistance, wherein representative seed of said Sorghum line has been deposited under ATCC Accession No. PTA-125106.

2. A plant part of the plant of claim 1.

3. The plant part of claim 2, further defined as pollen, an ovule, a tissue, a pod, a seed, and a cell.

4. A seed of Sorghum line BTX430-CHR-ACC1, wherein representative seed of Sorghum line BTX430-CHR-ACC1 has been deposited under ATCC Accession No. PTA-125106.

5. The seed of claim 4, further comprising a transgene, wherein the transgene was introduced into Sorghum line BTX430-CHR-ACC1 by backcrossing or genetic transformation.

6. A composition comprising the plant part of claim 2 in plant seed growth media, wherein representative seed of Sorghum line BTX430-CHR-ACC1 has been deposited under ATCC Accession No. PTA-125106.

7. The composition of claim 6, wherein the growth media is soil or other media.

8. An F1 hybrid seed produced by crossing a plant of Sorghum line BTX430-CHR-ACC1 according to claim 1 with a second, distinct Sorghum plant.

9. The F1 hybrid seed of claim 8 wherein said plant comprises ACC inhibitor herbicide tolerance that is inherited by the seed from BTX430-CHR-ACC1.

10. The F1 hybrid seed of claim 8, wherein said plant of Sorghum line BTX430-CHR-ACC1 further comprises a transgene that is inherited by the seed, wherein the transgene was introduced into Sorghum line BTX430-CHR-ACC1 by backcrossing or genetic transformation.

11. An F1 hybrid plant and/or a plant part thereof grown from the seed of claim 8.

12. A seed that produces the plant of claim 11.

13. The seed of claim 10, wherein the transgene confers a trait selected from the group of male sterility, herbicide tolerance, insect or pest resistance, disease resistance, and/or site- specific genetic recombination.

14. A method of producing a progeny ACC inhibitor pesticide resistant Sorghum plant, said method comprising applying plant breeding techniques to one or more plants with the ACC inhibitor pesticide resistance trait as present in BTX430-CHR-ACC1 representative seed deposited under ATCC Accession No. PTA-125106 to yield a progeny Sorghum plant, and thereafter selecting for ACC inhibitor pesticide resistance.

15. The method of claim 14 wherein said breeding techniques include selecting for ACC inhibitor pesticide resistance.

16. The method of claim 14, wherein the plant breeding techniques comprise backcrossing, marker assisted breeding, pedigree breeding, selfing, outcrossing, haploid production, doubled haploid production, gene editing, or transformation.

17. The method of claim 16, further defined as comprising: (a) crossing the plant of claim 1 or an F1 hybrid thereof with itself or a second plant to produce a seed of a progeny plant of a subsequent generation; (b) growing a progeny plant of a subsequent generation from said seed and crossing the progeny plant of a subsequent generation with itself or a second plant; and (c) repeating steps (a) and (b) with sufficient inbreeding until an inbred Sorghum plant is produced.

18. A method of producing a commodity plant product, said method comprising obtaining the plant of claim 1 or a part thereof or a progeny thereof and producing said commodity plant product therefrom.

19. The method of claim 19, wherein the commodity plant product is grain, starch, seed oil, Sorghum syrup, or protein.

20. A method of producing a Sorghum seed, the method comprising crossing two Sorghum plants and harvesting the resultant Sorghum seed, wherein at least one of the Sorghum plants is the Sorghum plant of claim 2.

21. A method comprising isolating nucleic acids from the plant, non-seed plant part, seed or plant cell of claim 2.

22. The method of claim 21 wherein said nucleic acid comprises one of the following:
the nucleotide sequence of SEQ ID NO: 2;
the nucleotide sequence of SEQ ID NO: 3;
the nucleotide sequence of SEQ ID NO: 4;
the nucleotide sequence of SEQ ID NO: 5;
the nucleotide sequence of SEQ ID NO: 2 and one of the following: the nucleotide sequence of SEQ ID NO: 3, SEQ ID NO: 4 or SEQ ID NO: 5;
the nucleotide sequence of SEQ ID NO: 3 and one of the following: the nucleotide sequence of SEQ ID NO:4 or SEQ ID NO: 5;
the nucleotide sequence of SEQ ID NO:4 and SEQ ID NO: 5;
the nucleotide sequence of SEQ ID NO: 2, SEQ ID NO: 3 and SEQ ID NO:4;
the nucleotide sequence of SEQ ID NO: 2, SEQ ID NO: 3 and SEQ ID NO: 5;
the nucleotide sequence of SEQ ID NO: 2, SEQ ID NO: 4 and SEQ ID NO: 5; or
the nucleotide sequence of SEQ ID NO: 3, SEQ ID NO: 4 and SEQ ID NO: 5.

23. The nucleic acid of claim 22 wherein the sequence encodes a Sorghum acetyl-CoA protein having a CT domain comprising one or more of the following mutations:
a Tryptophan to Cysteine amino acid substitution at an amino acid position 1999 (W1999C) aligning with the amino acid sequence of SEQ ID NO: 6, or
a Tryptophan to Serine amino acid substitution at an amino acid position 1999 (W19995) aligning with the amino acid sequence of SEQ ID NO: 6, or
an alanine to valine amino acid substitution at an amino acid position 2004 (A2004V) aligning with the amino acid sequence of SEQ ID NO: 6, or
a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 2027 W2027S) aligning with the amino acid sequence of SEQ ID NO: 6, or
a Tryptophan to Serine amino acid substitution at an amino acid position 1999 (W19995) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Serine amino acid substitution at an amino acid position 1999 (W19995) aligning with the amino acid sequence of SEQ ID NO: 6 and a Tryptophan to Serine amino acid substitution at an amino acid position 2027 W2027S) aligning with the amino acid sequence of SEQ ID NO: 6, or
an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Serine amino acid substitution at an amino acid position 1999 (W19995) aligning with the amino acid sequence of SEQ ID NO: 6 and an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6.

24. A method of plant breeding, to develop an ACC inhibitor herbicide tolerant plant variety comprising: (a) crossing a plant with ACC inhibitor herbicide tolerance as present in Sorghum line BTX430-CHR-ACC1 representative seed deposited under ATCC Accession No. PTA-125106 with a second plant that does not have ACC inhibitor herbicide tolerance; (b) selecting at least a first progeny plant from step (a) that comprises ACC inhibitor herbicide tolerance to produce a selected progeny plant; (c) crossing the selected progeny plant from step (b) with said second progeny plant to produce a backcross progeny plant that comprises ACC inhibitor herbicide tolerance.

25. The method of claim 24 wherein said ACC inhibitor tolerance is conference by a nucleic acid sequence comprising one of the following:
the nucleotide sequence of SEQ ID NO: 2;
the nucleotide sequence of SEQ ID NO: 3;
the nucleotide sequence of SEQ ID NO: 4;
the nucleotide sequence of SEQ ID NO: 5;
the nucleotide sequence of SEQ ID NO: 2 and one of the following: the nucleotide sequence of SEQ ID NO: 3, SEQ ID NO: 4 or SEQ ID NO: 5;
the nucleotide sequence of SEQ ID NO: 3 and one of the following: the nucleotide sequence of SEQ ID NO:4 or SEQ ID NO: 5;
the nucleotide sequence of SEQ ID NO:4 and SEQ ID NO: 5;
the nucleotide sequence of SEQ ID NO: 2, SEQ ID NO: 3 and SEQ ID NO:4;
the nucleotide sequence of SEQ ID NO: 2, SEQ ID NO: 3 and SEQ ID NO: 5;
the nucleotide sequence of SEQ ID NO: 2, SEQ ID NO: 4 and SEQ ID NO: 5; or
The nucleotide sequence of SEQ ID NO: 3, SEQ ID NO: 4 and SEQ ID NO: 5.

26. The nucleic acid of claim 25 wherein the sequence encodes a Sorghum acetyl-CoA protein having a CT domain comprising one or more of the following mutations:
a Tryptophan to Cysteine amino acid substitution at an amino acid position 1999 (W1999C) aligning with the amino acid sequence of SEQ ID NO: 6, or
a Tryptophan to Serine amino acid substitution at an amino acid position 1999 (W19995) aligning with the amino acid sequence of SEQ ID NO: 6, or
an alanine to valine amino acid substitution at an amino acid position 2004 (A2004V) aligning with the amino acid sequence of SEQ ID NO: 6, or
a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 2027 W2027S) aligning with the amino acid sequence of SEQ ID NO: 6, or
a Tryptophan to Serine amino acid substitution at an amino acid position 1999 (W19995) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Serine amino acid substitution at an amino acid position 1999 (W19995) aligning with the amino acid sequence of SEQ ID NO: 6 and a Tryptophan to Serine amino acid substitution at an amino acid position 2027 W2027S) aligning with the amino acid sequence of SEQ ID NO: 6, or
an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6 and an alanine to valine amino acid substitution at an amino acid position 2004 (A2004V) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and a Tryptophan to Serine amino acid substitution at an amino acid position 1999 (W1999S) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Cysteine amino acid substitution at an amino acid position 1999 (W1999C) aligning with the amino acid sequence of SEQ ID NO: 6 and an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6, or
a Tryptophan to Serine amino acid substitution at an amino acid position 1999 (W1999S) aligning with the amino acid sequence of SEQ ID NO: 6 and an alanine to valine amino acid substitution at an amino acid position 2004 (A2004V) aligning with the amino acid sequence of SEQ ID NO: 6 and a Tryptophan to Serine amino acid substitution at an amino acid position 2027 (W2027S) aligning with the amino acid sequence of SEQ ID NO: 6.
